(19) United States
(12) Patent Application Publication
(10) Pub. No.: US 2024/0252845 A1
(43) Pub. Date: Aug. 1, 2024

(54) TISSUE TREATMENT SYSTEM

(71) Applicant: Orchard Ultrasound Innovation LLC, Sunnyvale, CA (US)

(72) Inventors: Butrus T. Khuri-Yakub, Palo Alto, CA (US); Andre T. Khoury-Yacoub, Purchase, NY (US); John N. Irwin, III, Greenwich, CT (US); Stanley Yung Chuan Liu, Menlo Park, CA (US); Priscilla Marie Babb, Walnut, CA (US); R. Maxwell Flaherty, Topsfield, MA (US); J. Christopher Flaherty, Nottingham, NH (US)

(21) Appl. No.: 18/564,181

(22) PCT Filed: Jun. 1, 2022
(86) PCT No.: PCT/US22/31746

§ 371 Date: Nov. 27, 2023

Related U.S. Application Data
(60) Provisional application No. 63/195,292, filed on Jun. 1, 2021.
(60) Provisional application No. 63/286,161, filed on Dec. 6, 2021.

(51) Int. Cl. A61N 7/00

(52) U.S. Cl. CPC A61N 7/00

(57) ABSTRACT

A system for performing a medical procedure on a patient is provided. The system comprises at least one of: an energy delivery device: a chronic energy delivery device; and/or a force applying device. In particular, the system can be configured to treat and/or diagnose a patient suffering from sleep apnea. Methods and devices for treating sleep apnea and other medical conditions are also provided.

### RELATED APPLICATIONS

The present application claims priority to U.S. Provisional Patent Application Ser. No. 63/195,292 (Docket No. USD-004-PR1), titled “Tissue Interface System”, filed Jun. 1, 2021, the content of which is incorporated herein by reference in its entirety for all purposes.

The present application claims priority to United States Provisional Patent Application Ser. No. 63/286,161 (Docket No. USD-008-PR1), titled “Capacitive Micromachined Ultrasonic Transducer”, filed Dec. 6, 2021, the content of which is incorporated herein by reference in its entirety for all purposes.

This application is related to U.S. Provisional Application Ser. No. 62/728,616, (Docket No. USD-001-PR), titled “Medical Device with CMUT Array and Solid State Cooling, and Associated Methods and Systems—with Thermal Analysis”, filed Sep. 7, 2018, the content of which is incorporated by reference in its entirety for all purposes.

This application is related to U.S. application Ser. No. 16/130,896, (Docket no. USD-001-US), titled “Medical Device with CMUT Array and Solid State Cooling, and Associated Methods and Systems”, filed Sep. 13, 2018, U.S. Pat. No. 11,154,730, issued Oct. 26, 2021, the content of which is incorporated by reference in its entirety for all purposes.

This application is related to U.S. patent application Ser. No. 17/479,011 (Docket No. USD-001-US-CON1), titled “Medical Device with CMUT Array and Solid State Cooling, And Associated Methods and Systems”, filed Sep. 20, 2021, United States Publication Number US2022/0072338, published Mar. 10, 2022, the content of which is incorporated herein by reference in its entirety for all purposes.

This application is related to International PCT Patent Application Serial Number PCT/US2018/050943, (Docket No. USD-001-PCT), titled “Medical Device with CMUT Array and Solid State Cooling, and Associated Methods and Systems” filed Sep. 13, 2018, Publication Number WO 2019/055699, published Mar. 21, 2019, the content of which is incorporated by reference in its entirety for all purposes.

This application is related to U.S. Provisional Patent Application Ser. No. 63/126,078 (Docket No. USD-003-PR1), titled “Tissue Interface System”, filed Dec. 16, 2020, the content of which is incorporated herein by reference in its entirety for all purposes.

This application is related to International PCT Patent Application Serial Number PCT/US2021/063743, (Docket No. USD-003-PCT), titled “Tissue Interface System”, filed Dec. 16, 2021, Publication No. ______, published ______, the content of which is incorporated by reference in its entirety for all purposes.

### TECHNICAL FIELD

The embodiments disclosed herein relate generally to systems for performing a medical procedure on a patient, particularly systems that deliver ultrasound energy to perform a diagnostic and/or therapeutic procedure upon tissue of the patient.

### BACKGROUND

Numerous medical devices involve the transmission of energy into the patient in order to collect image data or treat tissue. There is a need for improved systems, devices, and methods for transmitting energy to diagnose or treat diseases and disorders of patients.

### SUMMARY

According to an aspect of the present inventive concepts, a system for performing a medical procedure on a patient comprises at least one of: an energy delivery device; a chronic energy delivery device; and/or a force applying device.

In some embodiments, the medical procedure comprises a diagnostic procedure, a therapeutic procedure, or both a diagnostic procedure and a therapeutic procedure.

In some embodiments, the system includes two or more of: the energy delivery device; the chronic energy delivery device; and/or the force applying device.

In some embodiments, the system is configured to diagnose and/or treat a sleep apnea patient. The system can include two or more of: the energy delivery device; the chronic energy delivery device; and/or the force applying device.

In some embodiments, the system is configured to ablate target tissue of the patient and to avoid damaging non-target tissue of the patient.

In some embodiments, the system comprises an array of ultrasound transducers comprising one, two, or more ultrasound transducers. The array of ultrasound transducers can comprise at least one piezo element, at least one CMUT element, and/or at least one piezo element and at least one CMUT element.

In some embodiments, the system further comprises a controller and a memory storage component coupled to the controller, and the memory storage component stores instructions to perform an algorithm. The algorithm can comprise an artificial intelligence algorithm. The algorithm can be configured to identify target tissue for ablation by the system. The algorithm can be configured to differentiate target tissue from non-target tissue.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a system for treating and/or diagnosing tissue, consistent with the present inventive concepts.

FIG. 2 is a flow chart of a method of providing a treatment in a closed-loop arrangement, consistent with the present inventive concepts.

FIG. 3 is a flow chart of a method for treating a patient, consistent with the present inventive concepts.

FIG. 4 illustrates a side sectional anatomic view of a chronic energy delivery device implanted in a patient for stimulating a nerve, consistent with the present inventive concepts.

FIG. 5 illustrates a side sectional anatomic view of a chronic energy delivery device implanted in a patient for stimulating a nerve, consistent with the present inventive concepts.

FIGS. 6A-B illustrate side sectional anatomic views of a force-applying device implanted in a patient for applying force to tissue, consistent with the present inventive concepts.

FIG. 7 illustrates a partially transparent anatomic view of a force-applying device implanted in a patient for applying force to tissue, consistent with the present inventive concepts.

FIG. 8 illustrates a sectional anatomic view of an energy delivery device delivering energy to tissue, consistent with the present inventive concepts.

FIG. 9 illustrates a side view of an energy delivery device delivering energy to tissue captured by the device, consistent with the present inventive concepts.

FIG. 10 illustrates a perspective view of an energy delivery device delivering energy to tissue captured by the device, consistent with the present inventive concepts.

FIG. 11 illustrates a perspective view of an energy delivery device delivering energy to tissue captured by the device, consistent with the present inventive concepts.

FIG. 12 illustrates a side sectional anatomic view of an energy delivery device which is positioned on the skin under the patient's chin and delivering energy to tongue tissue, consistent with the present inventive concepts.

FIG. 13 illustrates a side sectional anatomic view of an energy delivery device that has been advanced transnasally to position a transducer in the patient's airway, consistent with the present inventive concepts.

FIG. 14 illustrates a front anatomic view of an energy delivery device that has been positioned on the face of a patient, consistent with the present inventive concepts.

FIG. 15 illustrates a perspective view of a system including an energy delivery device with a shaft and a distally placed transducer whose diameter approximates that of the shaft, consistent with the present inventive concepts.

FIG. 16 illustrates a perspective view of a system including an energy delivery device with a shaft and a distally placed transducer whose diameter is larger than that of the shaft, consistent with the present inventive concepts.

FIG. 17 illustrates a side sectional anatomic view of an energy delivery device comprising an energy delivery module and a mirror, consistent with the present inventive concepts.

FIGS. 18A-B illustrate a top view, and a side sectional anatomic view of an energy delivery device, consistent with the present inventive concepts.

FIG. 19 illustrates a side sectional anatomic view of an energy delivery device that has been transnasally inserted into a patient, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

It will be understood that the words “comprising” (and any form of comprising, such as “comprise” and “comprises”), “having” (and any form of having, such as “have” and “has”), “including” (and any form of including, such as “includes” and “include”) and/or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of two or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, “prevention” and the like, where used herein, shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. “Positive pressure” includes pressure above room pressure or simply a pressure that is greater than another pressure, such as a positive differential pressure across a fluid pathway component such as a valve. “Negative pressure” includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “transducer” is to be taken to include any component or combination of components that receives energy or any input and produces an output. For example, a transducer can include an electrode that receives electrical energy and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising one or more piezoelectric and/or CMUT transducers configured to deliver and/or receive ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: heat energy to tissue; cryogenic energy to tissue; electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising one or more piezoelectric and/or CMUT transducers); chemical energy; electromagnetic energy; magnetic energy; and combinations of two or more of these. Alternatively or additionally, a transducer can comprise a mechanism, such as: a valve; a grasping element; an anchoring mechanism; an electrically-activated mechanism; a mechanically-activated mechanism; and/or a thermally activated mechanism.

As used herein, the term “functional element” is to be taken to include one or more elements constructed and arranged to perform a function. A functional element can comprise one or more sensors and/or one or more transducers. In some embodiments, a functional element is configured to deliver energy and/or otherwise treat tissue (e.g. a functional element configured as a treatment element). Alternatively or additionally, a functional element (e.g. comprising one or more sensors) can be configured to record one or more parameters, such as a patient physiologic parameter; a patient anatomical parameter (e.g. a tissue parameter); a patient environment parameter; and/or a system parameter (e.g. temperature and/or pressure within the system). In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a patient anatomical parameter; and combinations of two or more of these. A “functional assembly” can comprise an assembly constructed and arranged to perform a function, such as is described hereabove. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly. A functional assembly can comprise one or more functional elements.

As used herein, the term “agent” shall include but not be limited to one or more agents selected from the group consisting of: an agent configured to improve and/or maintain the health of a patient; a drug (e.g. a pharmaceutical drug); a hormone; a protein; a protein derivative; a small molecule; an antibody; an antibody derivative; an excipient; a reagent; a buffer; a vitamin; a nutraceutical; and combinations of these.

As used herein, the term “target tissue” comprises one or more volumes of tissue of a patient to be diagnosed and/or treated. Similarly, a “treatment target” or “tissue target” comprises one or more volumes of tissue to be diagnosed and/or treated. “Safety margin tissue” comprises tissue whose treatment (e.g. receiving of ablative energy) yields no significant adverse effect to the patient. “Non-target tissue” comprises tissue that is not intended to receive treatment (e.g. not intended to receive energy). In some embodiments, “target tissue”, “treatment target”, and/or “tissue target” comprises a non-tissue material, such as a pigment particle used in a tattoo, a splinter such as a wood or metal fragment, and/or other undesired material present in a patient's body.

As used herein, the term “system parameter” comprises one or more parameters of the system of the present inventive concepts. A system parameter can comprise one or more “energy delivery parameters” (also referred to as “energy delivery settings”), such as one, two or more energy delivery parameters selected from the group consisting of: energy form (e.g. ultrasound, light, electromagnetic, and the like); amplitude; frequency; waveform shape; a pulse width modulation parameter; a time division multiplexing parameter; pulse width; pulse rate; duty cycle; area of energy beam (e.g. ultrasound beam and/or light beam); location of energy delivery; tissue temperature, such as the starting temperature of tissue prior to treatment; other energy delivery parameters; and combinations of these. A system parameter can comprise a parameter selected from the group consisting of: an energy delivery parameter; a pressure level; a temperature level; an energy level; a frequency level; an amplitude level; a battery level; and combinations of these. A system parameter can include one or more tissue targets identified to be treated (e.g. volumes of tissue intended to be ablated and/or stimulated), such as tissue targets identified for treatment by an algorithm of the system.

As used herein, the term “patient parameter” comprises one or more parameters associated with the patient. A patient parameter can comprise a patient physiologic parameter, such as a physiologic parameter selected from the group consisting of: temperature (e.g. tissue temperature); pressure such as blood pressure or other body fluid pressure; pH; a blood gas parameter; blood glucose level; hormone level; heart rate; respiration rate; and combinations of these. Alternatively or additionally, a patient parameter can comprise a patient environment parameter, such as an environment parameter selected from the group consisting of: patient geographic location; temperature; pressure; humidity level; light level; time of day; and combinations of these.

As used herein, the term “image data” comprises data created by one or more imaging devices. Image data can include data related to target tissue, safety margin tissue, and non-target tissue. Image data can also include data related to any implants or other non-tissue objects that are proximate tissue being imaged. Image data can be processed by one or more algorithms of the present inventive concepts, such as to determine one or more locations to treat (e.g. target tissue identified to be ablated or otherwise receive energy), and/or to determine one or more locations to which energy delivery is to be avoided (e.g. non-target tissue). Image data can comprise data produced by a single imaging component, or from multiple imaging components.

As used herein, the term “transmitting a signal” and its derivatives shall refer to the transmission of power and/or data between two or more components, in any direction.

As used herein, the term “patient use data” shall refer to data related to use of the tissue interface system of the present inventive concepts on a patient (e.g. use of the system in a diagnostic and/or therapeutic procedure performed on a patient). The data can include but is not limited to: operating parameters such as energy delivery parameters, durations of energy delivery; target tissue parameters such as location of target tissue and/or amount of target tissue; patient parameters such as patient physiologic parameters and/or patient location or other patient environment parameters; clinician parameters; clinical site parameters; and combinations of these. Patient use data can include data from multiple patients, such as data collected from multiple patients that interface with one or more systems of the present inventive concepts. In some embodiments, an algorithm of the present inventive concepts uses patient use data from one or more patients to determine a system parameter to be used in performing a medical procedure on a patient.

As used herein, the term “conduit” or “conduits” can refer to an elongate component that can include one or more flexible and/or non-flexible filaments selected from the group consisting of: one, two or more wires or other electrical conductors (e.g. including an outer insulator); one, two or more wave guides; one, two, or more hollow tubes, such as hydraulic, pneumatic, and/or other fluid delivery tubes; one or more optical fibers; one two or more control cables and/or other mechanical linkages; one, two or more flex circuits; and combinations of these. A conduit can include a tube including multiple conduits positioned within the tube. A conduit can be configured to electrically, fluidically, sonically, optically, mechanically, and/or otherwise operably connect one component to another component.

As used herein, a component shall be considered an “implantable” component, or a component having been “implanted” in the patient, if the component is positioned at any location under the skin of the patient (e.g. within tissue of the patient, within and/or on an airway of the patient, and other internal locations). Implantable devices can be implanted in a patient via a surgical procedure (e.g. a procedure in which an incision is made in the patient's skin), and/or the devices can be implanted via delivery through a natural orifice (e.g. the mouth, eye, a nostril, ear canal, anal orifice, urinary meatus, vagina, and/or pores of the skin).

As used herein, an “ultrasound transducer” (also referred to as “ultrasound element”) can refer to one or more components configured to transmit ultrasound energy (e.g. based on a delivered electrical signal) and/or one or more components configured to receive ultrasound energy (e.g. and convert it to an electrical signal). An ultrasound transducer can comprise a set of one or more ultrasound transducers, such as a 1D or 2D array of ultrasound transducers. An ultrasound transducer can refer to: a set of one or more piezoelectric transducers (also referred to as “piezo” transducers or elements); a set of one or more capacitive micromachined ultrasound transducers (CMUTs), or a set of one or more of both.

As used herein, an “optical transducer” (also referred to as “optical element”) can refer to one or more components configured to transmit light (e.g. a diode such as a laser diode) and/or one or more components configured to receive and/or facilitate the travel of light (e.g. a lens, prism, optical fiber, and the like).

The systems of the present inventive concepts can be configured to deliver energy to treat and/or diagnose one or more medical conditions of a patient, using one or more energy delivery modules. The energy delivery modules of the present inventive concepts can comprise modules configured to deliver and/or receive various forms of energy, such as ultrasound energy. In some embodiments, an array of one, two, or more piezo transducers and/or an array of one, two, or more CMUTs are included to deliver ultrasound energy (e.g. to stimulate, ablate, and/or otherwise affect tissue), and/or to receive reflection of ultrasound energy (e.g. to image tissue, such as when the same and/or different elements deliver the energy that is reflected and received).

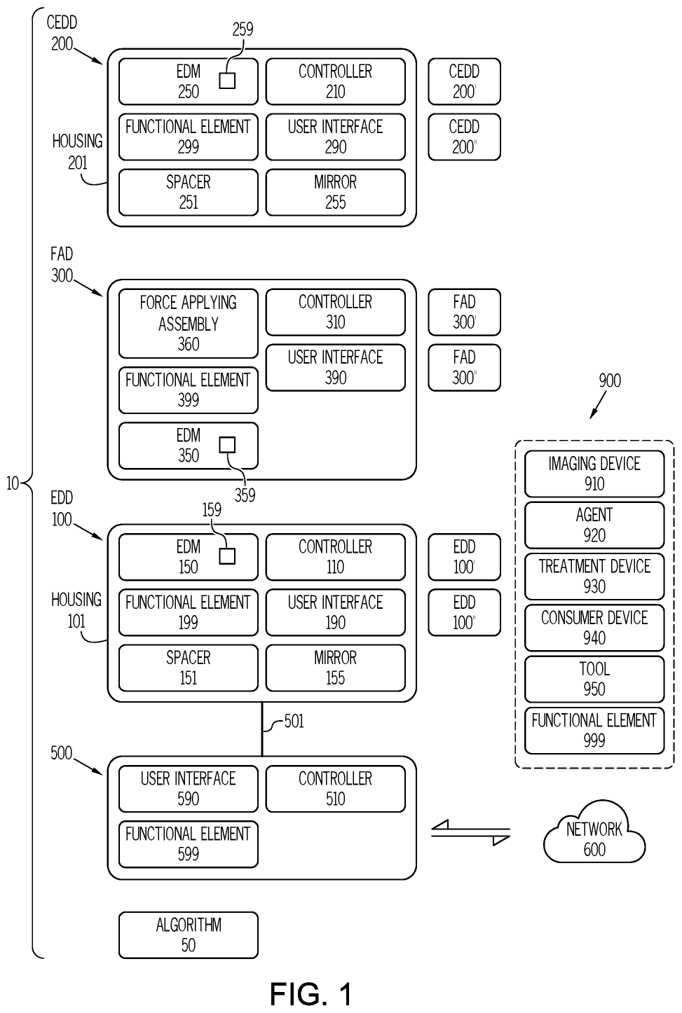

Referring now to FIG. 1, a schematic view of a tissue interface system is illustrated, consistent with the present inventive concepts. System 10 can be configured to perform a medical procedure on a patient. A medical procedure performed using system 10 can include the performance of one or more clinical procedures (also referred to as “medical procedures” herein), such as one or more diagnostic procedures and/or one or more treatment procedures performed on a patient. System 10 can be configured to diagnose and/or treat one or more medical conditions (e.g. diseases and/or disorders) of the patient. System 10 can be configured to treat and/or diagnose one or more portions (e.g. volumes) of patient tissue, “target tissue” herein. In some embodiments, system 10 comprises one or more devices that are configured to deliver one or more forms of energy to target tissue, such as to stimulate, modify, ablate, and/or otherwise treat the target tissue. Alternatively or additionally, system 10 can include one or more devices that are configured to produce image data, image data ID, which can comprise image data of tissue and/or one or more objects proximate tissue. Image data ID can include tissue or other object image data that is used in determining a diagnosis and/or prognosis (either or both “diagnosis” herein). Alternatively or additionally, image data ID can include tissue or other object image data that is used in a tissue treatment procedure (e.g. such as to guide or otherwise affect a stimulation, ablation, and/or other tissue treatment procedure). Image data ID can include image data related to: target tissue; safety margin tissue; non-target tissue; an implanted diagnostic and/or a treatment device; a foreign body (e.g. a splinter, tattoo, and the like); and combinations of these. System 10 can be configured to produce image data ID through the delivery of energy, such as sound energy and/or light energy that is delivered and whose reflections are collected in order to produce image data ID, as described herein. In some embodiments, image data ID comprises data related to tissue comprising blood, such as when image data ID comprises blood flow data (e.g. as obtained using Doppler ultrasound). In some embodiments, image data ID comprises tissue temperature information (e.g. one or more temperature readings for one or more volumes of tissue) and/or tissue ablation information (e.g. completion of ablation information for one or more volumes of tissue).

As used herein, a “tissue diagnostic procedure”, a “tissue diagnostic”, and their derivatives include but are not limited to: delivery of energy to collect image data ID (e.g. when system 10 records reflections of delivered ultrasound, light, or other energy, and converts these recordings into image data ID); delivery of energy to tissue to characterize the tissue (e.g. when system 10 records one or more effects on the tissue due to the energy delivery, such as using spectroscopy); and/or recording of one or more tissue properties using one or more sensors of system 10. In some embodiments, a tissue diagnostic performed by system 10 comprises a diagnosis of tissue, such as a diagnosis that assesses: the level of ablation of tissue (e.g. a volume of target tissue and/or non-target tissue); tissue temperature; tissue elasticity; tissue density; and/or tissue type (e.g. fat, nerve, muscle, and/or other tissue types in a volume of tissue). In some embodiments, system 10 utilizes a machine learning or other AI algorithm (e.g. as described herein) to perform a tissue diagnostic procedure (e.g. to assess level of ablation of tissue).

As used herein, a “tissue treatment procedure”, a “tissue treatment”, and their derivatives include but are not limited to: ablation of tissue; removal of tissue; causing the necrosis of tissue; reducing the volume of tissue (e.g. debulking tissue); stimulating tissue; improving the strength of tissue (e.g. muscle tissue); scaffolding of a tissue and/or an airway, manipulating and/or otherwise applying a force to tissue; stiffening tissue; and/or otherwise providing a therapeutic effect to tissue. As used herein, a “tissue reduction procedure” or a “tissue reduction”, and their derivatives, include a tissue treatment procedure in which a portion of tissue (e.g. target tissue) is reduced in volume, such as by: ablation of tissue, lithotripsy of tissue; histotripsy of tissue; removal of tissue, liquefication of tissue (e.g. liquefication of fat tissue); causing the necrosis of tissue; debulking tissue, and/or otherwise causing a volume of tissue to be reduced (e.g. reduced within 1 day, 1 week, and/or 1 month of treatment). In some embodiments, a tissue reduction procedure comprises a procedure that deforms tissue (e.g. deforms a geometry of a volume of tissue), such as to reduce the volume of tissue present in an airway, without necessarily reducing the overall volume of the tissue. As used herein, a “tissue enhancement procedure” or a “tissue enhancement”, and their derivatives, include a tissue treatment procedure in which a portion of tissue (e.g. muscle tissue or other tissue) is strengthened, stiffened, tightened, toned, moved (e.g. to a better location), and/or otherwise enhanced, such as an enhancement which is used to reduce one or more adverse effects of a disease or disorder of the patient (e.g. sleep apnea), and/or an enhancement used in a cosmetic procedure (e.g. to reduce wrinkles and/or otherwise improve patient cosmesis). A tissue enhancement procedure can include delivery of energy (e.g. ultrasound energy) to perform neuromodulation. A tissue enhancement procedure can include delivery of energy (e.g. ultrasound energy) configured to ablate certain tissue (e.g. tongue tissue) in order to strengthen neighboring muscle tissue (e.g. to strengthen muscle tissue of the tongue).

As used herein, a “treatment plan” comprises a set of parameters that are used in treating target tissue of the patient using system 10. A treatment plan can include a set of energy delivery settings, such as level or form of energy delivery, location of energy delivery, and/or other energy delivery parameter as defined herein. A treatment plan can include a set of different medical procedures (e.g. one or more of various medical procedures, such as tissue reduction procedures, tissue enhancement procedures, tissue force-applying procedures, pharmaceutical treatments, CPAP treatments, and/or other procedures). A treatment plan can include a desired and/or recommended order for performing a set of multiple medical procedures (e.g. where the treatment plan provides multiple procedures to be performed in a particular order, where in some instances sufficient efficacy is achieved when a subset of the procedures is performed). In some embodiments, system 10 is configured to automatically and/or semi-automatically (“automatically” herein) generate a treatment plan (e.g. one or more treatment plans made available to a clinician). System 10 can generate the treatment plan using an algorithm, such as algorithm 50 described herein. A treatment plan can be developed by algorithm 50 using at least image data ID, such as by using image data ID comprising: ultrasound-based image data (e.g. Doppler data and/or other image data produced using ultrasound); CT-based image data; MRI-based image data; and/or X-ray based image data (e.g. fluoroscopic data and/or other image data produced using X-ray). Alternatively or additionally, algorithm 50 can develop a proposed treatment plan based on parameters selected from the group consisting of: patient age; volume of target tissue to be ablated, reduced in volume, and/or otherwise treated (e.g. where target tissue comprises tumor tissue, adenoid tissue, tongue tissue, tonsil tissue, and/or other tissue); fatty content of target tissue; geometry of target tissue; tissue type, geometry and/or other characteristic of non-target tissue proximate the target tissue; geometry of an airway proximate target tissue; and combinations of these. In some embodiments, a treatment plan includes a methodology to ensure treatment of target tissue, while avoiding damage to neighboring non-target tissue. In some embodiments, system 10 (e.g. via algorithm 50) is configured to produce a prediction of outcome (e.g. an estimation of likelihood of efficacy and/or an assessment of any risks) associated with one or more treatment plans. For example, algorithm 50 can be configured to analyze data from a population of multiple patients, such as patients with a similar configuration of tissue (e.g. similar geometry of tissue related to influencing a sleep apnea event).

System 10 can include EDD 100, which can comprise one, two, or more energy delivery devices that are used by a doctor, nurse, and/or medical technician (“clinician” herein) to diagnose and/or treat a patient through the delivery of energy in a medical procedure. System 10 can include multiple energy delivery devices, such as EDD 100, 100′, and/or 100″ shown (generally EDD 100). During its medical use (e.g. diagnostic and/or therapeutic use), one or more energy delivery portions of EDD 100 is positioned at one or more locations on or otherwise proximate the patient, location L100 herein, such that target tissue proximate location L100 can be diagnosed and/or treated by EDD 100 via the delivery of one or more forms of energy, as described herein. In some embodiments, EDD 100 is configured to receive one or more forms of energy, such as reflected energy used to produce image data ID, and/or energy representing control signals or other data that has been sent (e.g. wirelessly sent) to EDD 100. EDD 100 can comprise a handheld device, a catheter, a probe (e.g. a probe configured to be inserted through a laparoscopic port), and/or a robotically controlled device. In some embodiments, EDD 100 comprises multiple discrete components. EDD 100 comprises one or more housings, housing 101 shown, such as one or more housings that surround one or more components of EDD 100.

System 10 can include CEDD 200, which can comprise one, two or more chronic energy delivery devices that are implanted in, positioned on, and/or otherwise provided to a patient such that a diagnosis and/or treatment can be performed on the patient on an ongoing basis (e.g. for a period of at least 1 week, at least 1 month, at least 3 months, and/or at least 6 months). System 10 can include multiple chronic energy delivery devices, such as CEDD 200, 200′, and/or 200″ shown (generally CEDD 200). During its medical use, one or more energy delivery portions of CEDD 200 is positioned at one or more locations within, on, and/or otherwise proximate the patient, location L200 herein, such that target tissue proximate location L200 can be diagnosed and/or treated by CEDD 200 via the delivery of one or more forms of energy, as described herein. Energy can be delivered by CEDD 200 on a relative continuous basis, and/or on an intermittent basis, also as described herein. In some embodiments, CEDD 200 is configured to receive one or more forms of energy, such as reflected energy used to produce image data ID, or energy representing control signals and/or other data that has been sent (e.g. wirelessly sent) to CEDD 200. CEDD 200 comprises one or more housings, housing 201 shown, such as one or more housings that surround one or more components of CEDD 200.

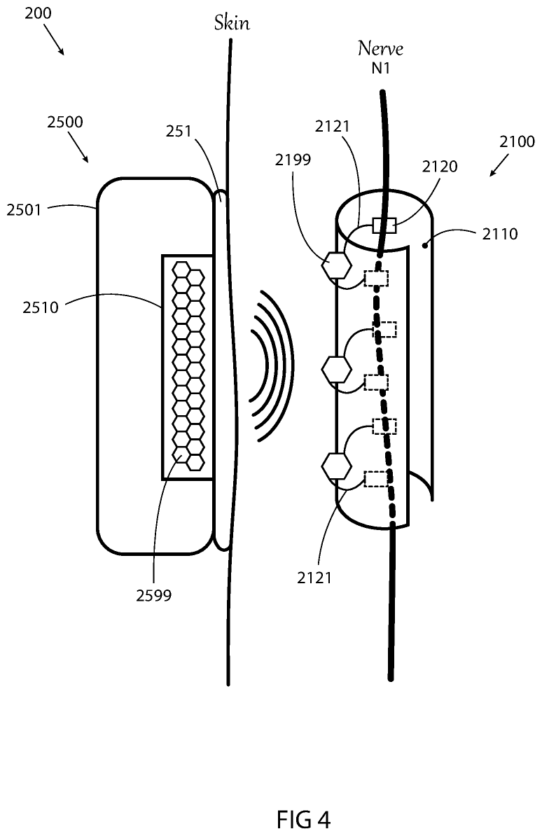

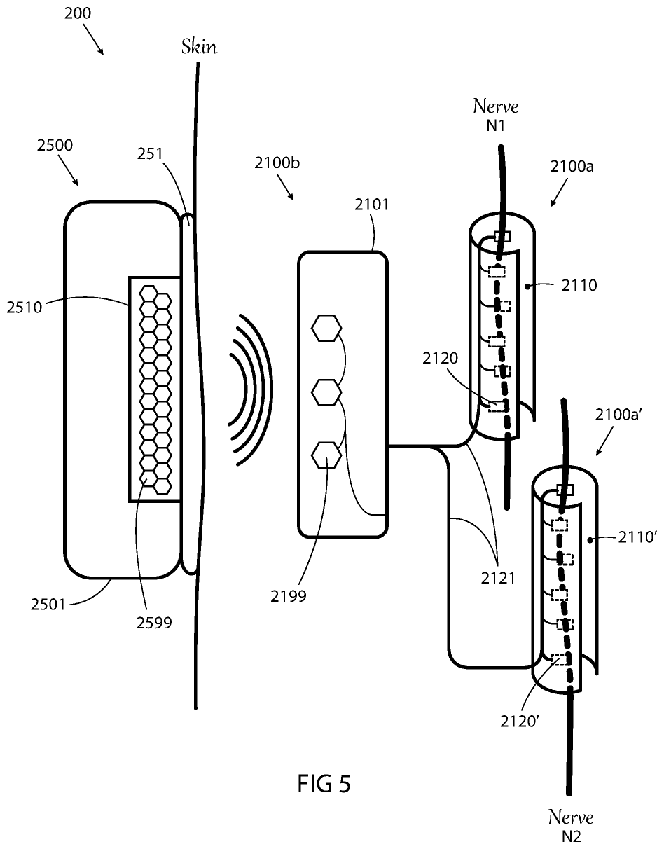

CEDD 200 can comprise one or more discrete components, such as one or more components that are positioned external but proximate the patient's skin during use, and/or one or more components that are implanted in (e.g. within an airway of) the patient. In some embodiments, CEDD 200 comprises a first component that includes EDM 250 and delivers energy to tissue (e.g. to perform an imaging procedure and/or a treatment procedure), and a second component that is configured to provide power to the first component, such as via a wired or wireless connection. For example, CEDD 200 can comprise a first component that is implanted in the patient, and a second component that (during use) is positioned on the skin of the patient proximate the implant location of the first component (e.g. as described in reference to FIG. 4 herein). In some embodiments, CEDD 200 can comprise a first component that is implanted in the patient, the first component comprising EDM 250 and configured to deliver energy to tissue (e.g. to perform an imaging procedure and/or a treatment procedure), and a second component that is also implanted in the patient, and is configured to provide power to the first component, such as via a wired or wireless connection (e.g. as described in reference to FIG. 5 herein). In these embodiments, CEDD 200 can include a third component that is positioned external to the patient, such as a third component that delivers power to the first component and/or the second component (e.g. via wireless energy transfer). CEDD 200 can comprise a component arranged as a flexible sheet and/or a tubular structure that can be wrapped around and/or positioned alongside a nerve, such as a component that comprises an array of piezo transducers and/or CMUTs (e.g. a tube and/or wrap of piezo transducers and/or CMUTs) that can receive ultrasound energy (e.g. from a second component of CEDD 200), convert the ultrasound energy to electrical energy that is in turn delivered to tissue (e.g. via one or more electrodes as described herein in reference to FIGS. 4-5). In these embodiments, an array of ultrasound transducers receiving the ultrasound energy can be configured as a relatively omnidirectional assembly, such that ultrasound energy can be successfully delivered from multiple locations (e.g. sensitivity to position of the component delivering the ultrasound energy is greatly reduced).

In some embodiments, CEDD 200 is configured to deliver energy (e.g. ultrasound energy) via EDM 250 to collect image data ID related to target tissue that also receives energy (e.g. stimulation energy) from EDM 250. The energy delivered by EDM 250 to produce image data ID (e.g. via reflections of the delivered energy received by EDM 250), and the stimulation energy delivered by EDM 250, can take the same pathway (e.g. the same pathway of ultrasound or other energy through the tissue). In these embodiments, the stimulation energy can be delivered in a closed-loop arrangement based on the image data ID collected (e.g. where EDM 250 switches continuously between imaging and stimulating modes). This closed-loop arrangement can provide enhanced efficacy of stimulation, for example when stimulating airway tissue to treat sleep apnea, where adjustments in the trajectory of stimulation energy delivery are made when the patient moves during sleep. In some embodiments, a first portion of EDM 250 at least produces image data ID (e.g. and optionally delivers stimulation energy), and a second portion of EDM 250 at least delivers stimulation energy (e.g. and optionally collects image data ID). In some embodiments, the first portion and the second portion of EDM 250 at least deliver stimulation energy to the same target tissue (e.g. such that the collective amount of stimulation energy delivered to target tissue is more than the maximum amount delivered by either portion of EDM 250).

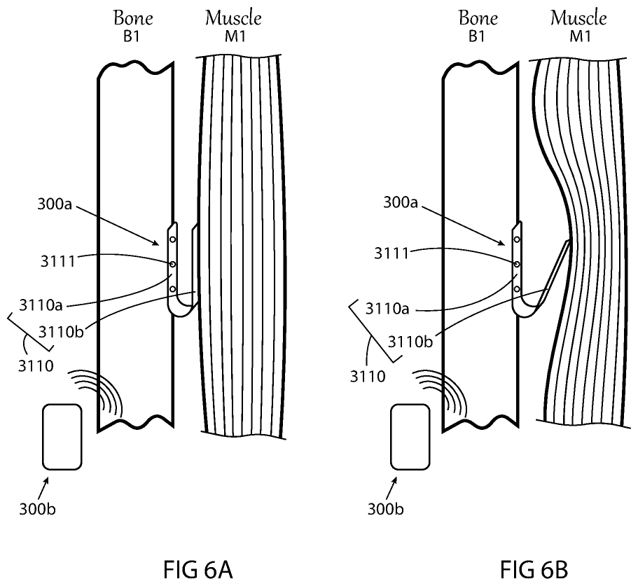

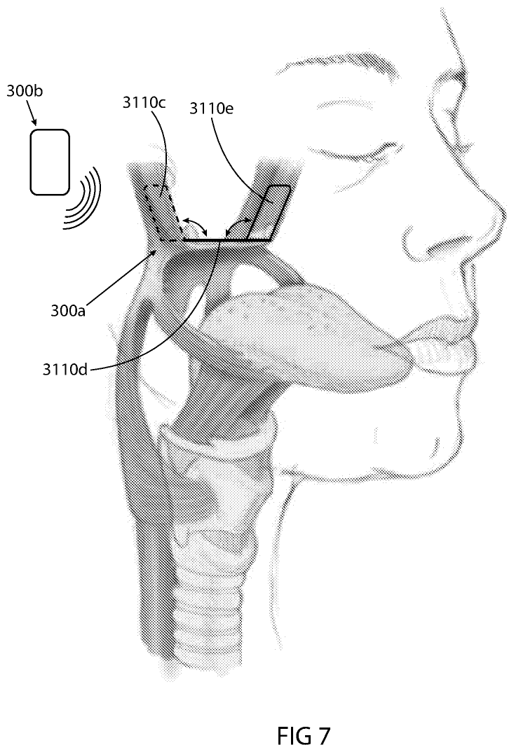

System 10 can include FAD 300 which can comprise one, two or more force-applying devices that are implanted in, positioned on, and/or otherwise provided to a patient such as to apply one or more forces upon target tissue of the patient (e.g. a force applied to tissue on a relatively continuous and/or intermittent basis for a period of at least 1 week, at least 1 month, at least 3 months, or at least 6 months). System 10 can include multiple force-applying devices, such as FAD 300, 300′, and/or 300″ shown (generally FAD 300). During its medical use, one or more force-applying portions of FAD 300 is positioned at one or more locations within, on, and/or otherwise proximate the patient, location L300 herein, such that target tissue proximate location L300 can receive the force applied by FAD 300. FAD 300 can be used to apply force to patient tissue (e.g. muscle tissue) to provide a therapeutic effect to that tissue (e.g. to continuously and/or intermittently exercise muscle tissue such as to strengthen the muscle). Alternatively or additionally, FAD 300 can be used to apply force to patient tissue to compress and/or otherwise scaffold the tissue (e.g. scaffold an airway), such as to increase the opening (e.g. the cross-sectional area) of an airway (e.g. to push tissue out of an airway of the patient, such as to treat sleep apnea). In some embodiments, FAD 300 is configured to apply a force to tissue of a passageway (e.g. a blood vessel, duct, tissue tube, and/or valve), such as to close the passageway (e.g. close a valve). FAD 300 can comprise one or more portions that attach to tissue (e.g. attaches to bone or other tissue), such as is described in reference to FIGS. 6A-B and/or 7 herein. FAD 300 can comprise an arch-like construction (e.g. including two actuator portions), such as is described in reference to FIG. 7 herein.

System 10 can include console 500 shown, which can comprise one or more discrete components which operably interface with one or more other components of system 10, such as to provide energy and/or data (e.g. control signals), and/or to receive energy and/or data. In some embodiments, console 500 comprises one or more components that are configured to operably attach to EDD 100, such as via a conduit, cable 501 shown, and/or via a wireless connection. In some embodiments, console 500 comprises one or more components that are configured to wirelessly communicate with other components of system 10, such as to transmit data to and/or receive data from EDD 100 (during use by a clinician in a medical procedure), CEDD 200 (e.g. when implanted in or positioned on the patient), and/or FAD 300 (e.g. when implanted in or positioned on the patient). Console 500 can comprise controller 510, which can include: one or more central processing units (CPUs), microprocessors and/or other microcontrollers; memory storage components (e.g. volatile or non-volatile memory); signal processing and other electronic circuitry, oscillator circuitry such as voltage-controlled oscillator (VCO) circuitry; analog to digital circuitry; digital to analog circuitry; and/or other componentry configured to control or otherwise interface with one or more components of system 10, such as EDD 100. Controller 510 can comprise a power supply and/or energy storage component (e.g. a battery and/or a capacitor). Controller 510 can comprise one or more electronic elements, electronic assemblies, and/or other electronic components, such as components selected from the group consisting of: memory storage components; analog-to-digital converters; rectification circuitry; state machines; microprocessors; microcontrollers; filters and other signal conditioners; sensor interface circuitry; transducer interface circuitry; and combinations thereof. In some embodiments, controller 510 comprises a memory storage component (e.g. coupled to controller 510), wherein the memory storage component includes instructions, such as instructions used by controller 510 to produce an energy delivery waveform and/or to perform an algorithm, each as described herein.

In some embodiments, console 500 comprises one, two, or more energy delivery assemblies, such as a controller 510 comprising one, two or more energy delivery assemblies. In these embodiments, controller 510 can comprise an assembly configured to deliver one or more forms of energy, such as energy selected from the group consisting of: ultrasound energy; radiofrequency and/or other electromagnetic energy; light energy (e.g. laser light energy); mechanical energy; chemical energy; thermal energy (e.g. heat energy and/or cryogenic energy); and combinations of these. In some embodiments, console 500 comprises a light source (e.g. a functional element 599 comprising a laser or other light source). The light source can be configured to provide light to EDD 100, CEDD 200, FAD 300, and/or another component of system 10. The light source can be configured to perform an imaging procedure (e.g. an OCT or other light-based imaging procedure), and/or perform a tissue elastography analysis. In some embodiments, a functional element 599 comprises a light source configured to deliver light to tissue and/or to deliver light to an agent, such as to affect the tissue and/or agent (e.g. to stimulate tissue and/or activate an agent, respectively). In some embodiments, controller 510 comprises one or more algorithms, such as algorithm 50 described herebelow. In these embodiments, energy delivery and/or another function provided by controller 510 and/or another component of console 500 can be controlled by the algorithm.

System 10 can comprise algorithm 50 shown, which can comprise one or more algorithms. All or a portion of algorithm 50 can be integrated into one, two, or more of various components of system 10, such as EDD 100, CEDD 200, FAD 300, and/or console 500. Algorithm 50 can comprise one or more machine learning, neural network, and/or other artificial intelligence algorithms (“AI algorithm” herein).

Algorithm 50 can be configured to determine and/or modify one or more energy delivery parameters, as defined herein, such as to effectively treat (e.g. ablate) target tissue and avoid damage to non-target tissue.

In some embodiments, algorithm 50 (e.g. an AI algorithm) can be configured to determine a volume of target tissue to be treated, such as to effectively provide a therapeutic benefit to the patient, while avoiding or at least minimizing damage to non-target tissue. In these embodiments, algorithm 50 can be further configured to determine and/or modify one or more energy delivery parameters (e.g. at least based on the determined volume), such as to effectively treat the target tissue volume determined, while avoiding damage to non-target tissue, as described hereabove.

In some embodiments, algorithm 50 is configured to perform a “tissue classification analysis” comprising a tissue ablation analysis (as described herebelow), a tissue type analysis (e.g. to differentiate fat, nerve, muscle, and other tissue types), and/or another form of tissue classification analysis. In these embodiments, the tissue classification analysis performed by algorithm 50 can be based on tissue elastography data, such as tissue elastography data collected by system 10, as described herein.

Algorithm 50 can be configured to perform a tissue classification analysis comprising a “tissue ablation analysis” comprising use of one or more types of information that is analyzed by algorithm 50 to assess the level of ablation (e.g. the current level of ablation) of target tissue (e.g. an ultrasound or MRI based elastography analysis that differentiates living tissue from dead tissue, and/or ablated tissue from non-ablated tissue). The results of this analysis can be used by system 10 to deliver energy in a closed-loop mode, as described herein. Tissue ablation data produced in the tissue ablation analysis can be stored as image data ID (e.g. and correlated with one or more tissue locations). In some embodiments, system 10 delivers and/or receives energy (e.g. ultrasound energy) to and/or from tissue, and algorithm 50 performs a tissue ablation analysis based on the delivered and/or received energy. The tissue ablation analysis can be configured to determine a size (e.g. the geometry of a volume of tissue) that has been ablated (e.g. sufficiently ablated to provide the intended benefit to the patient).

Algorithm 50 can be configured to perform a “tissue temperature analysis” comprising use of one or more types of information that is analyzed by algorithm 50 to assess the temperature (e.g. the current temperature) of tissue. The results of this analysis can be used by system 10 to deliver energy in a closed-loop mode, as described herein. Tissue temperature data produced in the tissue temperature analysis can be stored as image data ID (e.g. and correlated with one or more tissue locations). In some embodiments, system 10 delivers and/or receives energy (e.g. ultrasound energy) to and/or from tissue, and algorithm 50 performs a tissue temperature analysis based on the delivered and/or received energy. In some embodiments, system 10 includes an infrared camera assembly. For example, a functional element 199, 299, 399, 599, and/or 999 can comprise an infrared camera assembly configured to measure temperature of tissue (e.g. tissue being ablated), such as when system 10 is configured to perform closed-loop energy delivery based on the measured tissue temperature.

Algorithm 50 can be configured to adjust energy delivery parameters based on sensor signals, such as when sleep sensor signals are used to modify delivery of energy to a sleep apnea patient during sleep (e.g. to optimize the therapy provided to that sleep apnea patient). The adjustment of energy delivery can be performed during delivery of ablation energy, and/or delivery of stimulation energy.

In some embodiments, algorithm 50 is configured to confirm that signals produced by a sensor of system 10 are associated with the patient being treated by system 10, as described herein. For example, algorithm 50 can be configured to identify, differentiate, and/or confirm that snoring or other sounds recorded by system 10 are associated with the patient and not another person or other sound-producing source (e.g. a TV, pet, or other person in the room with the patient while the patient is sleeping).

In some embodiments, algorithm 50 is configured to receive signals from one or more sensors of system 10, and to identify whether the patient is breathing from their mouth or from their nose, such that system 10 can adjust delivery of energy accordingly.

In some embodiments, system 10 gathers image data ID using ultrasound (e.g. as described herein), such as B-mode collected ultrasound image data ID, and algorithm 50 can comprise an AI algorithm (e.g. a machine learning algorithm) configured to assess the image data ID to determine the level of ablation of tissue (e.g. determine if a volume of target tissue is sufficiently ablated).

In some embodiments, system 10 gathers image data ID (e.g. using ultrasound as described herein), and algorithm 50 analyzes the collected image data ID to create a treatment plan (e.g. as described herein), such as a treatment plan comprising one or more treatment plans that are provided to the clinician as suggestions for treatment of the patient from which image data ID was collected. In these embodiments, algorithm 50 can produce the treatment plan based on additional data, such as data related to any physiologic, genetic, and/or other patient information.

EDD 100, CEDD 200, and/or FAD 300 (singly or collectively “device 100/200/300” herein) can each include a control module, controllers 110, 210, and/or 310, respectively, as shown. Controllers 110, 210, and/or 310 (singly or collectively controller 110/210/310) can each include one or more central processing units (CPUs), microprocessors and/or other microcontrollers; memory (e.g. volatile or non-volatile memory); signal processing and other electronic circuitry; analog to digital circuitry; digital to analog circuitry; and/or other componentry configured to control or otherwise interface with one or more components of system 10, such as EDD 100, CEDD 200, and/or FAD 300, respectively. Controller 110/210/310 can comprise one or more electronic elements, electronic assemblies, and/or other electronic components, such as components selected from the group consisting of: memory storage components; analog-to-digital converters; rectification circuitry; state machines; microprocessors; microcontrollers; filters and other signal conditioners; sensor interface circuitry; transducer interface circuitry; and combinations thereof. In some embodiments, controller 110/210/310 comprises a memory storage component that includes instructions, such as instructions used by controller 110/210/310 to perform an algorithm, as described herein. Controller 110/210/310 can comprise a power supply, such as an energy storage component (e.g. a rechargeable battery and/or a capacitor). In some embodiments, at least a portion of a controller 110/210/310 is implanted in the patient, such as to adjust and/or otherwise control (e.g. automatically adjust via algorithm 50) the energy that is delivered by an implanted portion of the associated EDM 150/250/350 (e.g. without need for control signals being delivered by an external component of system 10). Alternatively or additionally, controller 110/210/310 can comprise at least a portion that is positioned external to the patient, and wirelessly delivers control signals to an implanted portion of EDD 100/200/300. In some embodiments, energy is wirelessly transferred to a battery, capacitor, and/or other energy storage element of an implanted portion of controller 110/210/310, such as via transmission of electrical energy (e.g. via inductive coupling and/or radiofrequency signals), and/or via transmission of ultrasound energy (e.g. ultrasound energy that is received by one or more ultrasound transducers and converted to electrical energy, as described herein). In these embodiments, the energy can be transferred from another implanted portion of controller 110/210/310, and/or from an externally placed portion of controller 110/210/310. In these embodiments, energy delivered to treat tissue (e.g. stimulate and/or ablate tissue) can be delivered by an implantable portion of EDM 150/250/350 at energy settings (e.g. frequency, amplitude, waveform shape, and the like) that are independent (e.g. different from) the energy settings of the energy delivery to the energy storage element of controller 110/210/310. For example, energy delivery to the implant can be at a higher frequency than the frequency of energy delivery used to treat tissue.

Controller 110/210/310 can comprise one or more energy storage components, such as a battery, capacitor, and/or other energy storage components. These one or more energy storage components can be positioned in an external portion and/or an implantable portion of devices 100/200/300 respectively. An energy storage component of controller 110/210/310 can be configured to be charged (e.g. recharged one or more times) via wireless power transmissions sent to controller 110/210/310, such as via a charging assembly (e.g. a tool 950 described herebelow comprising a charging assembly) configured to wirelessly charge an energy storage component of another device. In some embodiments, device 100/200/300 comprises a charging assembly (e.g. a wireless charger that is integrated into an external portion of device 100/200/300). In some embodiments, the wireless power transmission comprises electromagnetic energy that is received and stored by the controller 110/210/310 (e.g. received through patient tissue by a portion of an implanted controller 210 and/or 310). Alternatively or additionally, the wireless power transmissions can comprise transmission of sound energy (e.g. ultrasound energy), light energy, and/or other non-electromagnetic energy that is received by controller 110/210/310 (e.g. received through patient tissue by a portion of an implanted controller 210 and/or 310), converted to electrical energy (e.g. by an array of one or more ultrasound transducers, and/or optical transducers of CEDD 200 and/or FAD 300), and stored in an implanted energy storage component of controller 110/210/310. In some embodiments, an implanted energy storage component of a controller 210 and/or 310 is charged on a routine basis, prior to periods of use (e.g. delivery of a treatment to tissue). In some embodiments, recharging of a CEDD 200 or an FAD 300 can occur prior to an EDM 250 delivering therapeutic energy to tissue (e.g. stimulating energy) or prior to a force applying assembly, FAA 360 shown, applying therapeutic force to tissue, respectively. In these embodiments, recharging can occur prior to therapy delivery (e.g. recharging occurs prior to a nightly therapy that is delivered to a sleep apnea patient, as described herein).

Controller 110/210/310 can be configured in an automated mode, such as when energy delivered and/or force applied by a device 100, 200, and/or 300 is automatically adjusted (e.g. turned on, turned off, and/or changed in intensity and/or form). For example, system 10 can be configured to treat a sleep apnea patient, and a controller 110/210/310 can be configured to automatically perform a function selected from the group consisting of: turn on energy delivery and/or force delivery if the patient falls asleep (e.g. as determined by a sensor and/or algorithm 50 of system 10); turn off energy and/or force delivery if the patient wakes up (e.g. as determined by a sensor and/or algorithm 50 of system 10); change the energy and/or force delivery (e.g. increase and/or decrease energy and/or force delivery) if the occurrence of sleep apnea events changes (e.g. worsens and/or becomes more frequent, as determined by a sensor and/or algorithm 50 of system 10); and combinations of these. In some embodiments, system 10 is configured in a manual mode (e.g. with or without also being in an automated mode), wherein the patient can easily turn on, turn off, and/or modify energy and/or force delivery (singly or collectively, “energy delivery” or “force delivery” herein) of system 10, such as via voice control and/or a simple tap of a switch (e.g. via a user interface of system 10 as described herein). In some embodiments, if the patient turns off energy and/or force delivery manually (e.g. when the patient waked up), and system 10 subsequently determines that the patient has fallen asleep (e.g. fallen back asleep), system 10 automatically can turn back on energy and/or force delivery (e.g. to prevent a sleep apnea event from occurring).

Console 500 can include user interface 590, each as shown. User interface 590 can include various controls configured to receive input from an operator of system 10 (e.g. a clinician or other user of system 10), and it can include various output devices configured to provide information to an operator. User interface 590 can comprise one or more user input components selected from the group consisting of: button, switch, foot pedal, lever; keyboard; mouse; touchscreen; microphone; and combinations of these. User interface 590 can comprise one or more user output components selected from the group consisting of: display; touchscreen; light; speaker; tactile transducer; and combinations of these.

Similarly, EDD 100, CEDD 200, and/or FAD 300 can each include a user interface, user interfaces 190, 290, and/or 390, respectively, as shown, and each can include similar user input and/or user output components as described hereabove in reference to user interface 590. In some embodiments, at least a portion of user interface 590 of console 500 is integrated into one or more of user interfaces 190, 290, and/or 390. In some embodiments, user interface 290 and/or 390 is configured for use by an implanting clinician prior to the implantation of the associated device 200 and/or 300 into the patient (e.g. the user interface components comprise sealed components and/or are otherwise configured to be implanted in the patient after an initial use). User interfaces 190, 290, and/or 390 (singly or collectively “user interface 190/290/390” herein) can comprise one or more switches or other controls that allow a patient or other operator of system 10 to turn on, turn off, and/or adjust the delivery of force and/or energy delivered by a device 100/200/300 of system 10. In some embodiments, user interface 190/290/390, and/or 590, comprises a user interface that adapts (e.g. dynamically adapts), based on information collected by system 10 (e.g. image data ID and/or other patient information collected by system 10). For example, energy delivery options and/or other treatment parameters provided by the user interface can be modified (e.g. limited and/or expanded from a standard set of settings) based on data collected during a medical procedure performed using system 10 (e.g. energy delivery data, tissue ablation and/or other tissue characteristic data, and/or other data that is collected by one or more sensors of system 10, such as are described herein).

System 10 can comprise one or more functional elements, such as functional element 199 of EDD 100, functional element 299 of CEDD 200, functional element 399 of FAD 300, functional element 599 of console 500, and/or functional element 999, each as shown. Functional elements 199, 299, 399, 599, and/or 999 can each comprise one or more sensors and/or one or more transducers, as described herein. Functional elements 199, 299, 399, 599, and/or 999 can comprise a wireless element, such as a wireless sensor that can receive power wirelessly, and/or transmit signals wirelessly.

In some embodiments, functional elements 199, 299, 399, 599, and/or 999 comprise one or more sensors selected from the group consisting of: accelerometer; gravity-based sensor; strain gauge; acoustic sensor (e.g. a microphone or other acoustic sensor); electromagnetic sensor (e.g. a hall effect sensor); pressure sensor; vibration sensor; temperature sensor; vacuum sensor; GPS sensor; humidity sensor; flow sensor (e.g. an airflow sensor); and combinations of these.

In some embodiments, functional elements 199, 299, 399, 599, and/or 999 can comprise one, two, or more sensors configured to measure a “patient parameter” (e.g. a patient sleep parameter or other patient physiologic parameter, and/or a patient environment parameter, each as defined herebelow).

Functional elements 199, 299, 399, 599, and/or 999 can comprise a patient “physiologic sensor” comprising one, two, or more sensors configured to measure a patient “physiologic parameter” such as: a sleep parameter (e.g. as defined herebelow); heart rate; blood pressure; respiration rate; perspiration rate; blood gas level; blood glucose level; brain and/or other neural activity such as measured by electroencephalogram (EEG), local field potential (LFP), and/or neuronal firing (e.g. single neuron firing activity); eye motion; positive end-expiratory pressure (PEEP) sensor; physiologic parameters measured by a bone-positioned sensor (e.g. a jaw-positioned sensor, such as a sensor configured to measure snoring and/or other labored breathing, sleep level such as REM sleep level, presence of an apnea event, and/or other sleep parameter); and combinations of these.

Functional elements 199, 299, 399, 599, and/or 999 can comprise a “sleep sensor” comprising one, two, or more sensors configured to measure one or more “sleep parameters” of the patient, such as a parameter selected from the group consisting of: a snoring parameter (e.g. snoring amplitude, snoring frequency, snoring waveform shape, snoring type, and/or other snoring parameter); occurrence of a sleep apnea event; sleep state (e.g. stage 1, stage 2, stage 3, REM sleep); a breathing parameter (e.g. breathing type such as nostril breathing and/or mouth breathing, respiration rate, and/or other breathing parameter); patient position during sleep (e.g. on their left side, on their right side, on their back, and/or on their stomach); heart rate and/or heart variability; and combinations of these. In some embodiments, a sleep parameter comprises one, two, or more parameters that can be recorded (e.g. measured by one, two, or more sensors) and/or provided by a patient's wearable device, such as a smart watch, an activity-recording device (e.g. worn on the patient's wrist), and/or other portable device.

Functional elements 199, 299, 399, 599, and/or 999 can comprise a patient “environment sensor” comprising one, two, or more sensors configured to measure a patient “environment parameter” such as: room temperature; room pressure; room light level; room ambient noise level; room volume; and combinations of these.

In some embodiments, functional elements 199, 299, 399, 599, and/or 999 comprise one or more sensors configured to measure a system 10 parameter, such as a system parameter selected from the group consisting of: temperature (e.g. temperature of a portion of a system 10 component); velocity and/or acceleration; position; strain; energy delivery level; force delivery level; and combinations of these. In some embodiments, functional element 199, 299, 399, 599, and/or 999 comprise one or more sensors configured to measure a system parameter from multiple procedures performed using system 10 (e.g. multiple procedures performed on a single patient, and/or multiple patients). In these embodiments, algorithm 50 can be configured to analyze the recorded parameters (e.g. the recorded levels of the recorded parameters), such as to adjust system 10 (e.g. adjust one or more settings of system 10) based on the multiple procedure data analysis.

In some embodiments, functional elements 199, 299, 399, 599, and/or 999 comprise one or more transducers selected from the group consisting of: cooling element such as a Peltier element; heating element such as a Peltier element or a heat pump; vibrational transducer; light-producing element (e.g. a diode, laser, and/or other light-producing element); a light-receiving element (e.g. a photodetector, lens, filter, beam splitter, and/or other light-receiving element); a magnetic field-generating element; vacuum-generating element; a mechanical manipulator (e.g. a tissue manipulator and/or a system 10 component manipulator); a solenoid or other rotary or linear actuator; a motor; a drug or other agent delivery assembly; and combinations of these.

In some embodiments, functional elements 199, 299, 399, 599, and/or 999 comprise an assembly or other component configured to provide a vacuum to another component of system 10. For example, functional elements 199, 299, and/or 399 can comprise a tissue-engaging port configured to receive a vacuum (e.g. from controller 110, 210 and/or 310 and/or from console 500) and to stabilize tissue, capture tissue (e.g. draw tissue toward the port) and/or otherwise engage tissue, when the vacuum is applied to the port. Functional elements 199, 299, 399, 599, and/or 999 can comprise a source of vacuum, such as vacuum that can be applied to such a tissue-engaging port. In some embodiments, functional elements 199, 299, 399, 599, and/or 999 comprise a component configured to provide a vacuum to manipulate tissue, such as to move tissue relative to an EDM 150/250/350, such as to modify energy and/or force delivery to the tissue based on the tissue manipulation. Alternatively or additionally, the provided vacuum can be configured to position tissue (e.g. and maintain the position of the tissue) relative to EDM 150/250/350.

In some embodiments, functional elements 199, 299, 399, 599, and/or 999 comprise a mechanical manipulating assembly configured to manipulate (e.g. robotically manipulate) one or more components of system 10, such as to manipulate the position, configuration, and/or orientation of an EDM 150/250/350 relative to the patient. For example, the functional element can include a frame that is positioned proximate patient tissue, and an X-Y manipulator that moves EDM 150/250/350 in at least two dimensions relative to the patient tissue, such as to deliver energy from multiple locations based on the positioning performed by the manipulator within the frame.

In some embodiments, functional elements 199, 299, 399, 599, and/or 999 comprise an adhesive, and/or an adhesive dispensing component, such as when an adhesive is used to temporarily (e.g. less than 1 day) and/or chronically (e.g. at least 1 week, 1 month, or 3 months) attach a component of system 10 (e.g. a portion of device 100/200/300) to tissue of the patient, and/or to another component of system 10.

In some embodiments, functional elements 199, 299, 399, 599, and/or 999 comprise a cooling fluid or cooling component (e.g. a thermoelectric cooling element) and/or an assembly configured to provide cooling (e.g. provide cooling to a system 10 component). In some embodiments, system 10 is configured to provide cooling to tissue and/or a system 10 component during delivery of energy (e.g. ablative energy) such as to avoid damage to non-target tissue and/or to avoid degradation of a system 10 component. For example, system 10 can include a functional element comprising a cooling element positioned in a spacer 151 and/or 251 described herein. Alternatively or additionally, system 10 can comprise a functional element comprising an assembly configured to provide a cooling fluid (e.g. in a recirculating arrangement) to a spacer 151 and/or 251. In some embodiments, a cooling element is provided and positioned on a tissue surface to allow ablation of target tissue comprising subsurface tissue, while avoiding damage to the surface tissue proximate the subsurface tissue being ablated.

In some embodiments, functional elements 199, 299, 399, 599, and/or 999 comprise an assembly or other component configured to apply a force to tissue (e.g. a grasping component configured to place tissue in tension, and/or a pushing element configured to provide a compressive force to tissue), such as to apply a force (e.g. a tensioning and/or compressing force) to tissue (e.g. target tissue) while energy is being delivered to the target tissue by another component of system 10.

In some embodiments, functional elements 199, 299, 399, 599, and/or 999 comprise a position sensor (e.g. an accelerometer) that is configured to track the position of a system 10 component (e.g. EDM 150/250/350) and/or the patient (e.g. target tissue of the patient). In these embodiments, the patient and/or system 10 position information can be used to deliver energy and/or force in a closed-loop arrangement such as to vary the delivery based on motion of the patient and/or system 10 component (e.g. to redirect delivery and/or otherwise adjust delivery to compensate for the motion, such as via algorithm 50).

In some embodiments, functional elements 199, 299, 399, 599, and/or 999 comprise one or more fluid delivery elements (e.g. one or more needles) that are configured to deliver a material (e.g. a fluid and/or other flowable material), such as to a location proximate target tissue during energy delivery to the target tissue (e.g. to cool and/or warm that tissue). In some embodiments, the material delivered comprises agent 920 described herein (e.g. a pharmaceutical drug or other agent delivered to the patient).

In some embodiments, functional element 299 comprises a sensor configured to be positioned on the skin of the patient, and/or implanted within the patient, such as to measure a sleep parameter of the patient. In some embodiments, functional element 299 comprises a vibration sensor, an accelerometer, and/or another sensor that is positioned on or proximate the patient's jaw, such as to measure a snoring level or other sleep parameter of the patient.

In some embodiments, functional element 999 comprises a cell phone; a tablet; a computer (e.g. a laptop computer); an alarm clock; a bed shaker; an alert device; and combinations of these. Functional element 999 can comprise a cell phone, laptop, tablet, camera, and/or other patient-maintained device, that includes one, two, or more sensors, such as a sensor configured to measure a patient physiologic parameter (e.g. a sleep parameter) and/or a patient environment parameter, each as described herein. In some embodiments, functional element 999 comprises one, two, or more of these sensors (e.g. patient physiologic and/or patient environment sensors) that are positioned in imaging device 910, treatment device 930, consumer device 940, and/or tool 950, each as described herein.

System 10 can include accessory device 900 shown, which can include one or more accessory devices to be used to treat and/or diagnose a patient. In some embodiments, accessory device 900 comprises one or more imaging devices, imaging device 910 shown. Imaging device 910 can comprise one, two, or more imaging devices selected from the group consisting of: magnetic resonance imager (MRI); Ct scanner; ultrasound imager; OCT and/or other laser-based imager; fluoroscope or other X-ray based imager; and combinations of these. In some embodiments, imaging device 910 provides image data ID to system 10, such that one or more of devices 100/200/300 can be guided via the provided image data ID. For example, imaging device 910 can comprise an MRI or ultrasound imager that provides image data ID configured to direct (e.g. automatically and/or manually direct) energy delivery (e.g. focused ultrasound and/or other energy delivery) to tissue by EDM 150 and/or EDM 250, and/or to direct (e.g. automatically and/or manually direct) application of force to tissue by FAA 360. In some embodiments, imaging device 910 comprises an ultrasound imager that provides positive end-expiratory pressure (PEEP) measurements, such as for quantifying and/or otherwise monitoring effects of therapy provided by system 10 as described herein. In some embodiments, a device 100/200/300 is constructed and arranged to include imaging device 910 (e.g. device 100/200/300 comprises an array of ultrasound elements and/or optical elements that are configured to produce image data ID via transmission and/or receiving of ultrasound energy and/or light energy, respectively, as described herein).

In some embodiments, accessory device 900 comprises one or more agents, agent 920 shown. Agent 920 can comprise one, two, or more agents selected from the group consisting of: a pharmaceutical drug; a cooling agent; a warming agent; a lubricating agent; a conductive agent such as an acoustically, electrically, and/or thermally conductive gel; a magnetic agent; a visualizable agent such as a radiographic, magnetic, and/or ultrasonically reflective agent; and combinations of these.

In some embodiments, accessory device 900 comprises one or more treatment devices, such as treatment device 930 shown. In some embodiments, treatment device 930 comprises an implant configured to be inserted into the soft palate or other airway tissue of the patient, such as to stiffen and/or reduce vibration of that tissue, and/or to cause deflection, compression, and/or other repositioning of that tissue out of the airway (e.g. to reduce the likelihood of a sleep apnea event). In some embodiments, treatment device 930 comprises a stent, such as a temporary or permanent stent placed in a segment of the nasal cavity and/or other patient airway segment to influence the shape of that segment (e.g. after a treatment of system 10 is performed in or proximate that segment, as described herein). In some embodiments, treatment device 930 comprises a distraction device, such as a jaw distraction device. In some embodiments, treatment device 930 comprises a surgical tool such as a surgical tool selected from the group consisting of: scalpel; an electrosurgical dissection and/or hemostasis device (also referred to as a “bovie”); retractor; clamp; fastening device such as suture, staple, adhesive, and the like; and combinations of these. In some embodiments, algorithm 50 is configured to control treatment device 930 in an automated or semi-automated (“automated” herein). For example, energy delivery and/or other treatment provided by treatment device 930 can be provided in a closed-loop arrangement, via algorithm 50. In some embodiments, algorithm 50 controls the operation of treatment device 930 based on an analysis of image data ID collected by system 10 (e.g. ultrasound-based image data ID as described herein). The image data ID used by algorithm 50 to control treatment device 930 can be collected by system 10 just prior to the controlling of treatment device 930, such as image data ID that is collected (e.g. at least a portion is collected) within 5 minutes, within 3 minutes, and/or within 1 minute of the control of treatment device 930 by algorithm 50.

In some embodiments, accessory device 900 comprises one or more customized or standard consumer electronic devices, such as consumer device 940 shown. In some embodiments, consumer device 940 comprises one, two or more devices selected from the group consisting of: cell phone (e.g. a cell phone); watch (e.g. a smart watch); a camera; an alarm clock; a computer such as a laptop computer or tablet; a bed such as an electrically adjustable bed; a night table device such as a clock and/or a lamp; and combinations of these.

In some embodiments, accessory device 900 comprises one or more tools, tool 950 shown. Tool 950 can comprise one, two, or more components selected from the group consisting of: a bed, such as an electrically adjustable bed; a bed shaker; an alarm clock or other bedside device (e.g. configured to wirelessly communicate with a device 100/200/300); and combinations of these. In some embodiments, tool 950 comprises a tool that adjusts the patient's position (e.g. an adjustable bed or other tool) to reduce patient snoring and/or to reduce the likelihood of an apnea event, such as an adjustment that is initiated and/or otherwise controlled via a signal provided by algorithm 50 (e.g. an algorithm 50 configured to detect and/or predict snoring and/or an apnea event). In some embodiments, tool 950 comprises a charging device such as a wireless charging device configured to deliver electromagnetic, ultrasound, light, and/or other energy to a separate device in order to charge that device. In these embodiments, device 100/200/300 can include tool 950 (e.g. a charging device is integrated into device 100/200/300). In some embodiments, tool 950 can comprise an assembly configured to stabilize one or more components of system 10, such as to maintain the position of EDM 150 of EDD 100 relative to patient tissue (e.g. to allow some patient movement during energy delivery while prevent undesired delivery of energy to non-target tissue). In these embodiments, tool 950 can comprise a tool that can be attached to the patient's jaw, and/or a tool that can maintain the patient's jaw in a particular position and/or prevents motion of the patient's jaw. Tool 950 can comprise a face mask assembly, a retainer, or both. In some embodiments, tool 950 is customized for a particular patient, such as when tool 950 is manufactured and/or otherwise configured based on a photograph of the patient, and/or a medical image (e.g. an ultrasound image, x-ray, or other medical image, such as a 2D or 3D image produced by a scanning device) of the patient's tissue. Tool 950 can comprise a manipulating tool, which can be configured to manipulate an EDM 150/250/350 relative to patient tissue, as described herein. In some embodiments, tool 950 is configured to position EDM 150/250/350 in one, two, or three dimensions relative to target tissue (e.g. target tissue of the tongue).

Accessory device 900 can comprise functional element 999, such as is described hereabove and otherwise herein. In some embodiments, consumer device 940 comprises functional elements 999, such as when consumer device 940 comprises a bed or other device positioned proximate the patient during sleep, and functional element 999 comprises one or more sensors configured to measure a patient parameter such as a patient sleep parameter.

System 10 can include network 600 as shown, which can comprise one, two, or more wired and/or wireless computer networks such as the Internet, a local area network, cellular network, and/or other data sharing, storage, and/or transmitting platform. Network 600 can be configured to transfer data between two or more system 10 components, and/or between a system 10 at a first location (e.g. a first hospital or other clinical setting) and a system 10 located at a second location (e.g. a second hospital or other clinical setting). Network 600 can comprise a network that is accessed by a component of system 10 when system 10 is configured to transfer information in a cloud-based arrangement. Network 600 can be used to transfer and/or store patient information and/or system 10 use information, such as when algorithm 50 (e.g. an AI algorithm) is configured to analyze information from a single patient and/or from groups of multiple patients in order to adjust the use of system 10 for those or other patients (e.g. via creation of a treatment plan as described herein).

EDD 100 and/or CEDD 200 (singly or collectively “device 100/200” herein) can each include an energy delivery module, EDM 150 and/or EDM 250, respectively, as shown. In some embodiments, FAD 300 comprises an energy delivery module, EDM 350 as shown, such that FAD 300 can both apply a force to tissue as well as deliver energy to tissue (e.g. deliver energy to image and/or ablate tissue). EDM 150, 250, and/or 350 (singly or collectively “EDM 150/250/350” herein) can be configured to deliver one, two, or more forms of energy that are delivered to tissue to perform a tissue diagnostic procedure and/or a tissue treatment procedure, as described above and otherwise herein. EDM 150/250/350 can be configured to deliver and/or receive energy to and/or from tissue, such as to generate image data ID of the tissue, such as when energy (e.g. ultrasound energy and/or light energy) is delivered by at least a portion of EDM 150/250/350, and reflected energy (e.g. reflected ultrasound energy and/or light energy, respectively) is received by a similar and/or a different portion of EDM 150/250/350, such that image data can be created based on the transmitted and received energies. In some embodiments, system 10 is configured to produce image data ID that includes tissue temperature information (e.g. one or more temperature readings for one or more volumes of tissue) and/or tissue ablation information (e.g. completion of ablation information for one or more volumes of tissue), as described herein. For example, algorithm 50 can be configured to determine tissue temperature information and/or tissue ablation information based on other image data ID produced via energy delivered and/or received by EDM 150/250/350, also as described herein. In some embodiments, EDM 150, 250, and/or 350 comprise an array of energy delivering and/or energy receiving components, energy delivery elements 159, 259, and/or 359 respectively, such as an array of one or more piezoelectric elements and/or one or more CMUTs that is configured to deliver and/or receive ultrasound energy. In some embodiments, EDM 150/250/350 is configured to be controlled (e.g. in a closed loop or other automated arrangement) via algorithm 50 (e.g. an AI or other algorithm configured to control EDM 150/250/350 based on image data ID and/or other data collected by system 10). In some embodiments, EDM 150/250/350 is of similar construction and arrangement to those described in applicant's co-pending International PCT Patent Application Serial Number PCT/US2021/063743, titled “Tissue Interface System”, and filed Dec. 16, 2021 [Docket No. USD-003-PCT].

EDD 100 and/or CEDD 200 can each include a component configured to reflect energy, such as mirror 155 and/or 255 respectively, as shown. Mirror 155 and/or 255 (singly or collectively “mirror 155/255” herein) can each comprise one or more mirrors, such as one or more acoustic mirrors (e.g. a mirror configured to reflect ultrasound energy, such as to redirect the ultrasound energy toward target tissue). In some embodiments, mirror 155/255 comprises a mirror configured to reflect light energy and/or another form of energy. Mirror 155/255 can be constructed and arranged, and/or positioned at a tissue location such that energy delivered is directed (or redirected) toward target tissue. Mirror 155/255 can be positioned temporarily (e.g. less than 1 day) and/or chronically (e.g. at least 1 week, 1 month, or 3 months) within the patient.

FAD 300 can include force-applying assembly, FAA 360, as shown. FAA 360 can comprise one, two, three, or more controllable actuator portions (“actuators” herein), such as when including an actuator that bows, curls, cantilevers, rotates, and/or otherwise adjusts its shape based on an applied voltage and/or current, temperature change, and/or other drive signal of FAD 300. In some embodiments FAA 360 comprises a first actuator that applies force to a first tissue location (e.g. a location comprising a first tissue type, such as muscle), and at least a second actuator that applies force to a second tissue location (e.g. a location comprising the same type of tissue and/or a different type of tissue). FAA 360 can comprise three or more actuators. In some embodiments, FAA 360 comprises multiple actuators that are independently deployable (e.g. as controlled by controller 310), such as to be deployed in different ways (e.g. different shapes and/or different forces applied to tissue) to optimize therapy (e.g. via an optimization procedure in which the deployment of the actuators is titrated or otherwise optimized for a particular patient's anatomy and/or other physiologic parameters specific to that patient).

FAA 360 can comprise a piezo-based actuator that changes shape based on a drive signal (e.g. an applied voltage). FAA 360 can comprise a shaped memory alloy and/or a shaped memory polymer that causes a shape change in FAA 360 when its temperature changes (e.g. via heating from a delivered current and/or cooling from a thermoelectric cooler and/or other cooling element). FAA 360 can comprise an electromechanical assembly comprising motors, gears, actuators, cams, and/or other components that can be remotely controlled (e.g. via controller 310) such as to transition between an undeployed state and a deployed (e.g. force-applying) state.

In some embodiments, FAA 360 comprises one, two, or more bimorph actuators, such as a piezo bimorph actuator comprising a first layer that contracts and a second layer that expands when a voltage is applied to the actuator. Alternatively or additionally, FAA 360 can comprise one, two, or more unimorph actuators (e.g. a piezo actuator configured as a cantilever comprising one active layer and one inactive layer).

FAD 300, via controller 310, can provide a drive signal to FAA 360 that comprises an alternating current (AC) and/or a direct current (DC) drive signal. An AC drive signal can be delivered to FAA 360 (e.g. to a piezo actuator as described hereabove) that causes a back and forth movement, such as to apply a varying force to tissue that provides a function selected from the group consisting of: tone, strengthen, and/or otherwise enhance muscle tissue (e.g. muscle tissue of an airway of the patient); apply a force to tissue of an airway that accommodates patient breathing (e.g. an AC signal that is synchronized with patient respiration). The amplitude of the AC drive signal can correlate to a distance of deflection and/or the force applied to tissue by FAD 300. A DC drive signal can be delivered to FAA 360 such as a DC signal that is applied continuously, intermittently, or both. Similarly, the magnitude (e.g. voltage level) of the DC signal correlates to a distance of deflection and/or the force level applied to tissue by FAD 300. A DC signal can be applied to create a fixed forced applied to tissue, and/or a fixed shape adjustment (e.g. fixed distance of actuation).

FAA 360 can be configured to apply an adjustable force (e.g. by adjusting the magnitudes of an AC or DC signal), such that the force can be adjusted in a procedure configured to optimize patient treatment, such as a force titration procedure performed where patient feedback is collected (e.g. comfort feedback) and/or one or more patient physiologic parameters are monitored and analyzed (e.g. manually by the clinician and/or automatically by algorithm 50). The force applied by FAA 360 can be adjusted continuously and/or intermittently, such as in a closed-loop mode as described herein. In some embodiments, forces applied by multiple actuators of FAA 360 are titrated independently and/or as a set.

FAA 360 (and/or other portions of FAD 300) can include one or more portions that are positioned on and/or within tissue, such as on and/or within airway tissue, muscle tissue, and/or other tissue to be scaffolded and/or enhanced (e.g. strengthened). FAA 360 can be anchored to bone or other tissue, such as in one, two, or more locations. FAA 360 can be anchored to bone or other tissue using screws, staples, suture, and/or other anchoring elements. In some embodiments, FAA 360 and/or FAD 300 comprises an arch-like construction (as shown in FIG. 7) that can be anchored in tissue via its shape, without needing separate anchoring elements. In these embodiments, FAA 360 and/or FAD 300 can be inserted and removed multiple times (e.g. on a regular basis).

FAA 360 can be driven by an AC drive signal of relatively low frequency and/or a DC signal, and it can have a thickness of a few millimeters. FAD 300 can comprise a first portion 300a that is implanted and includes FAA 360, the implanted first portion 300a receiving power from a second portion 300b (e.g. a second implanted portion or a portion positioned on the skin of the patient, proximate the implanted first portion). The power delivered by portion 300b can comprise an ultrasound energy transmitted at a frequency of a few MHz (e.g. a resonant frequency of the FAD 300 thickness of a few millimeters). The first implanted portion of FAD 300 can harvest the received ultrasound energy (e.g. via one or more piezo transducers and/or CMUTs), converting it into a DC signal (e.g. DC power used immediately), and/or storing it in a capacitor and/or other energy storage element. Because of the relatively large size of FAA 360, FAA 360 can be “directional” at the frequency of the power delivery. In some embodiments, second portion 300b comprises ultrasound elements arranged as a focused array (e.g. large aperture) configured to deliver focused ultrasound to a receiving set of ultrasound elements of first portion 300a. The ultrasound elements of second portion 300b can comprise a 2D array of ultrasound elements that deliver power to first portion 300a.

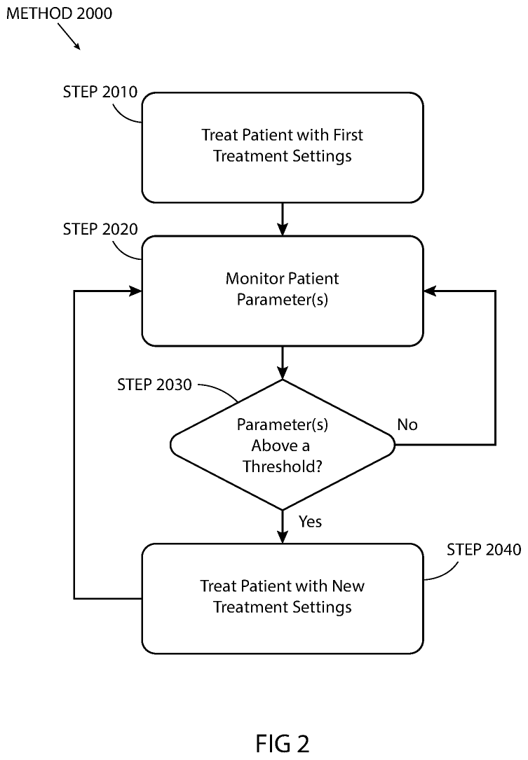

In some embodiments, FAA 360 is constructed and arranged as described in reference to FIGS. 2, 6A-B, and/or 7 herein.

In some embodiments, EDD 100 and/or CEDD 200 comprise a force-applying assembly, not shown, but of similar construction and arrangement as assembly 360 of FAD 300 described herein.

EDD 100, CEDD 200, and/or FAD 300, such as via EDM 150 (e.g. via an array of one, two, or more elements 159), EDM 250 (e.g. via an array of one, two, or more elements 259), and/or EDM 350 (e.g. via an array of one, two, or more elements 359), respectively, and/or another component of system 10, can be configured to deliver energy of one, two, or more energy forms, such as one, two, or more energy forms selected from the group consisting of: sound energy such as high intensity focused ultrasound (HIFU) energy, other focused ultrasound energy, planar wave ultrasound energy, and/or other ultrasound energy; light energy such as laser light energy; electromagnetic energy such as radiofrequency (RF) energy and/or microwave energy; thermal energy such as heat energy and/or cryogenic energy; mechanical energy; chemical energy; and combinations of these. In some embodiments, console 500 delivers one or more of these forms of energy to a component of system 10 (e.g. to EDD 100 when it is operably attached to console 500). EDM 150/250/350 can be configured to deliver energy (e.g. ultrasound and/or other sound energy, light energy, electrical energy, and/or another form of energy) to perform a tissue reduction procedure (e.g. a tissue ablation procedure, a lithotripsy procedure, and/or a histotripsy procedure) and/or to perform a tissue enhancement procedure (e.g. a nerve or muscle stimulation procedure) on the patient.

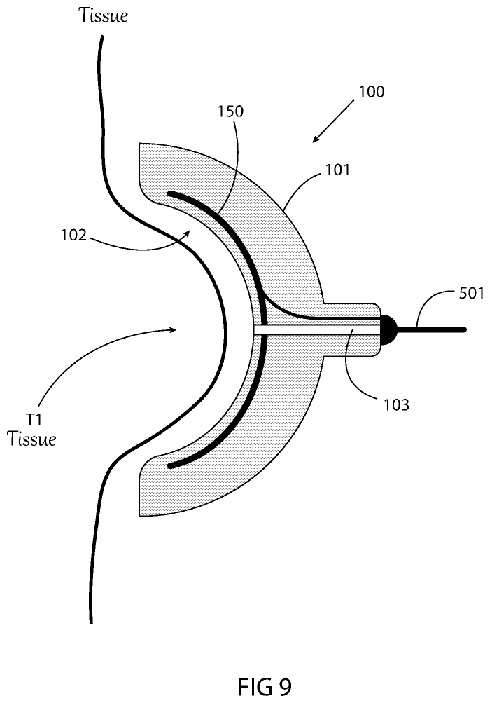

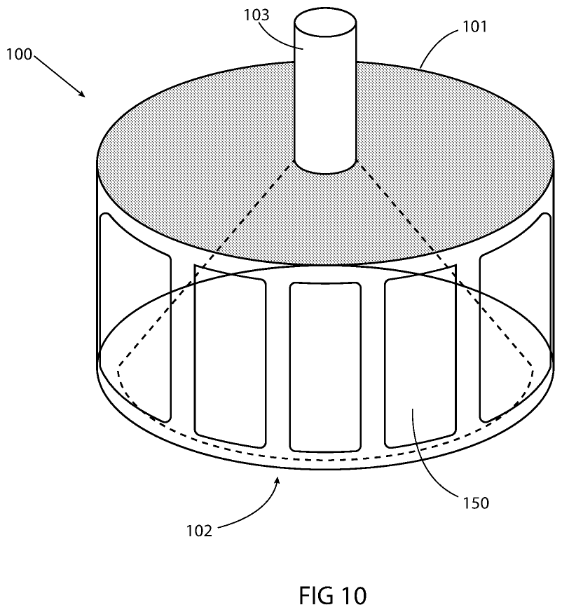

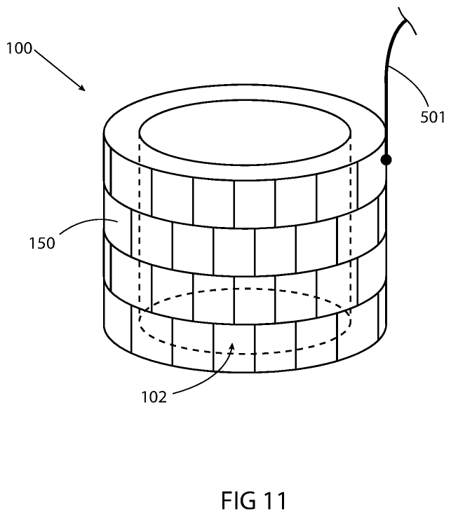

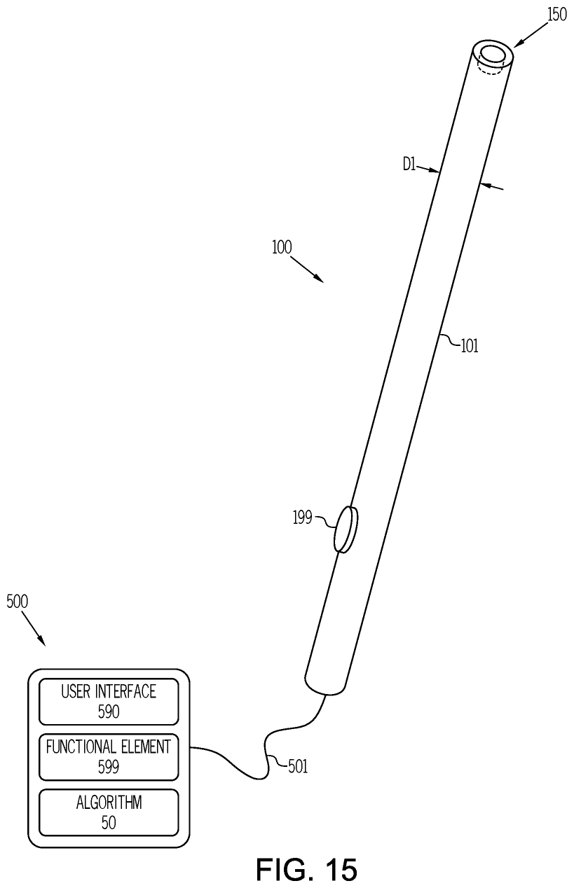

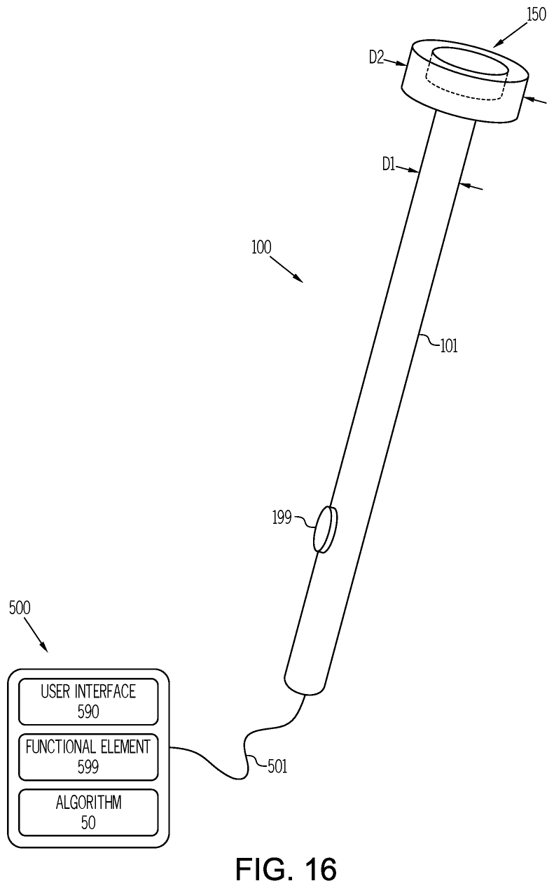

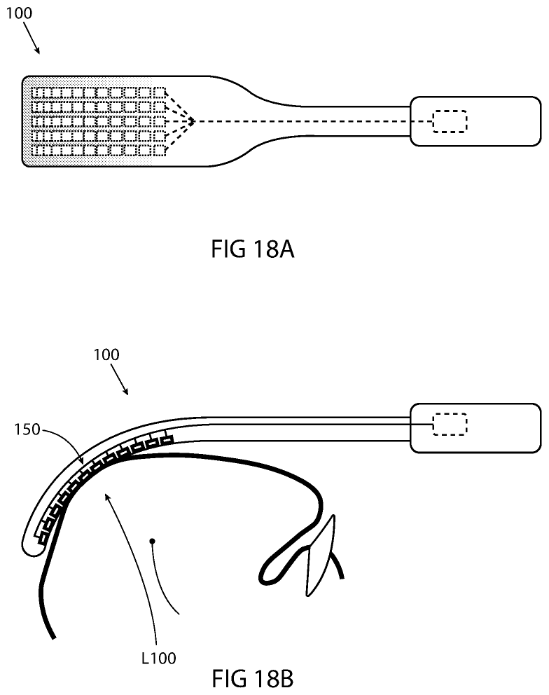

EDM 150/250/350 can comprise one or more energy delivery elements 159, 259, and/or 359, respectively that are used to collect image data, such as by receiving reflected energy (e.g. energy reflections from energy delivered by the same or other elements). Elements 159, 259, and/or 359 (singly or collectively elements 159/259/359) can comprise a 1D or 2D array of elements (e.g. ultrasound elements). Elements 159/259/359 can comprise a horizontal and/or vertical arrangement of elements. Elements 159/259/359 can comprise an array of elements (e.g. ultrasound elements) configured in an arrangement selected from the group consisting of: multiple 1D arrays of elements; a flat array of elements; a curved array of elements; an arrangement of elements in the form of a flexible wrap (e.g. as described in reference to FIGS. 4 and/or 5 herein); an array of elements positioned on the end of an elongate probe and comprising a diameter approximating the diameter of the probe (e.g. as described in reference to FIG. 15 herein); an array of elements positioned on the end of an elongate probe and comprising a diameter larger than the diameter of the probe (e.g. as described in reference to FIG. 16 herein); an array with a spoon shape or other convex and/or concave shape (e.g. to sufficiently contact the surface of the tongue of a patient such as to deliver energy to subsurface tissue of the tongue, such as is described in reference to FIGS. 18A-B herein); a tubular array of elements (e.g. a solid or hollow tubular construction); a partial circumferential array of elements; and combinations thereof. In some embodiments, EDM 150/250/350 can comprise a geometry (e.g. a geometry including elements 159/259/359) that is configured to hold and/or otherwise stabilize tissue that is to receive energy from the elements 159/259/359, such as an arrangement comprising a cup (e.g. as described in reference to FIG. 9 herein), a cone (e.g. as described in reference to FIG. 10 herein), and/or a cylinder (e.g. a full or partial circumferential cylinder, such as is described in reference to FIG. 11 herein). Alternatively or additionally, EDM 150/250/350 can comprise one or more vacuum ports (e.g. functional elements 199, 299, and/or 399, respectively, comprising one or more vacuum ports) that is configured to stabilize tissue (e.g. as described in reference to FIGS. 9, 10, and/or 11 herein). In some embodiments, device 100/200/300 can be configured to compress the tissue that is to receive energy (e.g. energy intended to ablate and/or stimulate), such as when device 100 is manipulated by an operator and/or a component of system 10 (e.g. a robotic manipulator) to compress tissue prior to and/or during delivery of energy by EDM 150/250/350 to the tissue.

In some embodiments, EDM 150/250/350, and/or another portion of device 100/200/300, comprises a geometry that is adjustable, such as an adjustable diameter of a cup, cone, and/or cylinder of an array of elements 159/259/359 (e.g. as described in reference to FIGS. 9, 10, and 11 herebelow). In these embodiments, an operator can adjust the geometry to adjust to characteristics of tissue to be treated (e.g. adjust to accommodate the dimensional characteristics of the patient's tonsil, tongue, and/or other tissue to be treated). In some embodiments, one or more portions of device 100/200/300 comprise a geometry (e.g. an adjustable geometry), such that force (e.g. an adjustable force) can be applied to target tissue, such as a tensile force and/or a compressive force that is applied to target tissue (e.g. target tissue to be ablated). In some embodiments, system 10 includes a kit of multiple devices 100/200/300 and/or components EDM 150/250/350 (e.g. attachable versions) which can have various geometries (e.g. various dimensions of tissue contacting portions of those components, such as to apply a desired force and/or otherwise accommodate the geometry of the tissue to be treated). In these embodiments, a clinician can choose from a kit of components a component that will optimize treatment (e.g. optimize energy delivery in tissue imaging and/or treatment).

EDM 150/250/350 can comprise an array of ultrasound transducers that deliver ultrasound energy (e.g. via a set of all or a portion of the array of ultrasound transducers) at or above a minimum frequency (e.g. a frequency at or above 10 MHz, such as a frequency between 10 MHz and 20 MHz) to create image data ID (e.g. image tissue and/or other objects on and/or within the patient), while also delivering ultrasound energy (e.g. via the same or different set of ultrasound transducers) at or below a maximum frequency (e.g. a frequency at or below 10 MHz, or below 5 MHz) to ablate tissue. For example, EDM 150/250/350 can image tissue with ultrasound delivered at or above 10 MHz, and ablate tissue via HIFU energy delivery at a frequency less than 10 MHz, such as less than 5 MHz.

EDM 150/250/350 can comprise an array of ultrasound transducers (e.g. CMUT transducers) that receive (e.g. and deliver) ultrasound energy in a “collapsed state” during creation of image data ID, and deliver energy at an ablative level using ultrasound transducers (e.g. all or some of the transducers used to create image data ID) that are in a “non-collapsed” state.

EDM 150/250/350 can comprise one or more energy delivery elements 159, 259, and/or 359, respectively that are used to collect image data, such as by receiving reflected energy (e.g. energy reflections from energy delivered by the same or other elements). Elements 159, 259, and/or 359 (singly or collectively elements 159/259/359) can comprise a 1D or 2D array of elements (e.g. ultrasound elements). Elements 159/259/359 can comprise an array of elements (e.g. ultrasound elements) configured in an arrangement selected from the group consisting of: multiple 1D arrays of elements; a flat array of elements; a curved array of elements; an arrangement of elements in the form of a flexible wrap (e.g. as described in reference to FIGS. 4 and/or 5 herein).

EDM 150/250/350 can comprise an array of one, two, or more ultrasound transducers that cause thermal ablation via delivery of focused ultrasound (e.g. HIFU). EDM 150/250/350 can comprise an array of one, two, or more ultrasound transducers that are configured to perform lithotripsy and/or histotripsy. EDM 150/250/350 can comprise an array of one, two, or more ultrasound transducers that deliver ultrasound energy to heat tissue to a non-ablative level, such as to cause a temperature increase that is below 50° C., or to cause a temperature increase between 43ºC and 50° C. that is of limited duration (e.g. a duration of no more than 2 minutes). In these non-ablative ultrasound energy deliveries, EDM/150/250/350 can be configured to liquefy certain types of tissue (e.g. fat tissue) while avoiding damage to other tissue types (e.g. muscle and/or nerve tissue). Alternatively or additionally, EDM 150/250/350 can be configured to deliver ultrasound energy at a non-ablative level that provides a “heat-massaging effect” to tissue, such as to tighten and/or otherwise enhance tissue such as muscle tissue.

EDM 150/250/350 can comprise an array of ultrasound transducers that are configured to deliver ultrasound energy at a frequency that can be adjusted (e.g. by a clinician and/or automatically by system 10). For example, EDM 150/250/350 can be configured to deliver ultrasound energy (e.g. planar wave delivery of ultrasound energy) to target tissue comprising a volume of tissue with a particular thickness, such as to ablate from a tissue surface proximate the ultrasound array to a maximum depth from that array, where the depth of ablation is dependent on the frequency of ultrasound energy delivered (e.g. controlled depth ablation of tissue).

EDM 150/250/350 can comprise an array of ultrasound transducers (e.g. a 2D array of ultrasound introducers) that are configured to deliver focused ultrasound energy to ablate multiple tissue locations (e.g. simultaneously or sequentially), such as multiple tissue locations that are positioned within a volume of tissue that has a non-homogenous structure (e.g. different tissue properties such as tissue type, tissue echogenicity, tissue cooling and/or other thermal properties, and the like). The multiple tissue locations to be ablated can comprise multiple locations within the tongue, tonsil, and/or other airway location. In these embodiments, EDM 150/250/350 can comprise an array of ultrasound transducers that deliver HIFU and/or other focused ultrasound at a frequency between 5-10 MHz. The multiple tissue locations can each comprise a relatively small volume of tissue, such as a volume with length and width of approximately 0.5 mm by 3.0 mm, or 1 mm by 6.0 mm. In some embodiments, multiple relatively small volumes of tissue are ablated, such as to avoid significant swelling that would occur if a larger volume of tissue was ablated.

EDM 150/250/350 can comprise an array of ultrasound transducers that are configured to deliver ablative ultrasound energy to ablate tissue, while avoiding the creation of a scab, such as when the ultrasound energy is delivered to subsurface tissue while avoiding adverse effects to surface tissue, as described herein.

EDM 105/250/350 and/or imaging device 910 can collect image data ID that includes data related to target tissue and/or non-target tissue (e.g. data that can be used to differentiate target tissue and non-target tissue, and/or to differentiate tissue types such as muscle, fat, nerve, and the like). Image data ID comprising these two forms of data can be used manually by an operator of system 10, and/or automatically by algorithm 50 to determine one or more energy delivery settings, as described herein, such as energy delivery settings that cause ablation of target tissue without damage to non-target tissue. In some embodiments, image data ID can comprise data related to a level of ablation or other damage to tissue, as described herein. In these embodiments, algorithm 50 can be configured to stop delivery of energy, such as a stoppage that occurs when sufficient ablation of target tissue is confirmed (e.g. to avoid damage to neighboring tissue), and/or when damage (e.g. any damage) of non-target tissue is detected. In some embodiments, the algorithm comprises a bias, such as a bias to tend to avoid damage to non-target tissue (e.g. a bias that potentially allows a portion of target tissue to be unablated or otherwise untreated), or a bias to tend to ensure all target tissue is ablated or otherwise treated (e.g. a bias that potentially allows a portion of non-target tissue to be ablated or otherwise damaged).

EDM 150/250/350 and/or imaging device 910 can collect image data ID that includes data related to a blood conduit of the patient, such as an artery, vein, and/or chamber of the heart. Image data ID comprising blood conduit data can be used to avoid (e.g. manually by an operator and/or automatically via algorithm 50) delivery of ablative energy to the blood conduit (e.g. the blood conduit and the blood within it is non-target tissue). Alternatively or additionally, image data ID comprising blood conduit data can be used to cause (e.g. manually by an operator and/or automatically via algorithm 50) the ablation of the blood conduit (e.g. to cause subsequent death of tissue fed by that particular blood conduit). In some embodiments, image data ID comprises data related to air or other gas pockets within the patient, such as gas pockets (e.g. as identified by algorithm 50 and/or an operator) that are to be avoided in the delivery of energy (e.g. ultrasound energy) by EDM 150/250/350, For example, in some embodiments, the trajectory of energy delivery from EDM 150/250/350 to target tissue to be ablated should avoid the gas pockets. Avoidance of the gas pockets using image data ID can be performed manually by an operator of system 10, and/or automatically by algorithm 50.

In some embodiments, EDM 150, 250, and/or 350 is configured to deliver ultrasound energy to tissue, such as focused ultrasound (e.g. HIFU or other focused ultrasound) and/or unfocused ultrasound energy delivery. In these embodiments, EDM 150/250/350 can comprise an array of energy delivery elements (e.g. elements 159 for EDM 150, element 259 for EDM 250, and/or element 359 for EDM 350) that comprise an array (e.g. a 1D or 2D array) of: one, two, or more piezo transducers; one, two, or more CMUTs; and/or at least one piezo transducer and at least one CMUT. In some embodiments, EDM 150/250/350 comprises an ultrasound-based array including both one or more piezo transducers, and one or more CMUT transducers. In these embodiments, the piezo transducers can perform one or more functions that are not performed by the CMUT transducers, or the CMUT transducers can perform one or more functions that are not performed by the piezo transducers. A particular one or more other functions can be performed by both the one or more piezo transducers as well as the one or more CMUT transducers. Algorithm 50 can be configured to compare the results of a function performed by both piezo and CMUT transducers (e.g. an imaging function), such as to differentiate the two results (e.g. and utilize results from one versus the other), and/or to combine the results to produce a new result set based on both piezo and CMUT data. In some embodiments, ultrasound imaging is performed in which piezo transducers emit ultrasound waves, and CMUT transducers receive the reflections of the emitted waves (e.g. reflections from various surfaces of tissues or other objects). Ultrasound energy delivered by EDM 150/250/350 can be configured to perform a tissue reduction procedure and/or a tissue enhancement procedure as described herein. In some embodiments, ultrasound energy delivered by EDM 150/250/350 can be received by a separate component of system 10 (e.g. an implanted or other portion of EDM 150/250/350), where the separate component converts the received ultrasound energy into electrical energy, such as electrical energy that is delivered to stimulate and/or otherwise treat tissue (e.g. via a voltage applied by one or more electrodes, such as is described herein in reference to FIGS. 4-5).

EDM 150, 250, and/or 350 can be configured to deliver energy (e.g. ultrasound energy) at a relatively low power density, such as to stimulate tissue without adversely affecting the tissue (e.g. avoiding cell death, such as to stimulate the tissue without adversely affecting the cells of the tissue). Alternatively or additionally, EDM 150/250/350 can be configured to deliver energy (e.g. ultrasound energy) with energy delivery settings that cause cell death, such as via heating at a level and for a duration that causes cell death, or at a level that results in histotripsy (e.g. controlled cavitation of tissue). For example, each device 100, 200, and/or 300 described herein can be configured to allow an operator of system 10 (e.g. via a user interface of system 10), to transition between the device delivering a tissue enhancement treatment (e.g. stimulation) and that device delivering a tissue reduction treatment (e.g. thermal ablation and/or histotripsy). System 10 can be configured to allow adjustment (e.g. manual or automatic) between continued and pulsed delivery of energy, as well as adjustment of the frequency of energy delivery (e.g. frequency of ultrasound energy to be delivered).

EDM 150, 250, and/or 350 can be configured to deliver ultrasound energy to tissue, as described herein. Ultrasound energy can be delivered by EDM 150/250/350 to stimulate tissue (e.g. nerve tissue), such as when the ultrasound energy is delivered at a power density of less than 50 W/cm². Ultrasound energy can be delivered by EDM 150/250/350 in a HIFU arrangement, such as when energy is delivered at a power density of at least 100 W/cm². In some embodiments, EDM 150/250/350 are configured to perform histotripsy on tissue, such as when a pressure level of 50 MPa is delivered.

EDM 150, 250, and/or 350 can comprise an assembly including one or more Peltier elements that are configured to heat and cool target tissue (e.g. where tissue on one side of a Peltier element is cooled and tissue on the other side of a Peltier element is heated), such as to exercise tissue, strengthen tissue, and/or massage tissue.

EDD 100 and/or CEDD 200 can each include a spacing element, spacer 151 and/or spacer 251 respectively. In some embodiments, FAD 300 includes a spacing element, not shown but of similar construction and arrangement as spacer 151 and/or 152 described herein. Spacer 151 and/or 251 (singly or collectively “spacer 151/251” herein) can comprise an element configured to be positioned between EDM 150 and/or EDM 250 and a tissue surface, respectively, when EDD 100 and/or CEDD 200 is delivering energy to target tissue. Spacers 151/251 can comprise a balloon, reservoir, and/or other fluid-containing structure configured to expand and/or contract when fluid is added and/or removed, respectively. In some embodiments, spacer 151/251 comprises water or other sound-conducting material (e.g. with an impedance that approximates the impedance of tissue) such that ultrasound delivered from EDM 150 and/or EDM 250 (singly or collectively “EDM 150/250” herein) passes through spacer 151/251 and into the patient in a predictable manner. In some embodiments, spacer 151/251 has an adjustable thickness, such as to allow an operator to adjust (e.g. manually adjust) the distance between EDM 150/250 and a tissue surface, and/or to allow EDD 100, CEDD 200 and/or another component of system 10 to adjust (e.g. automatically adjust) the distance between EDM 150/250 and a tissue surface. System 10 can be configured to automatically adjust the thickness of spacer 151/251, such as via an AI-based algorithm 50. Spacer 151/251 can be temporarily or permanently attached to a housing of device 100 and/or 200. In some embodiments, spacer 151/251 is configured to removably attach (e.g. adhesively attach) to the patient's skin (e.g. on one side of spacer 151/251), and/or to removably attach (e.g. adhesively attach) to a housing of device 100 and/or 200 (e.g. on the opposite side of the spacer). Spacer 151/251 can comprise a visual grid on a surface, such as to guide attachment of a housing of device 100 and/or 200 at multiple locations. In some embodiments, spacer 151/251 can be configured to provide a cooling function, such as to extract heat from EDM 150/250 and/or from tissue of the patient. In some embodiments, spacer 151/251 comprises an electrode, such as a return electrode used to enable delivery of monopolar electrical energy by electrodes of devices 100 and/or 200 (e.g. electrodes 2120 described herebelow).

In some embodiments, one or more components of system 10 include at least a “resorbable” portion, in other words a portion that is configured to be implanted in the patient and safely degrade over time. In some embodiments, at least a portion of: CEDD 200, FAD 300 (e.g. at least a portion of FAA 360), and/or treatment device 930 (e.g. a stent-type or other scaffolding-type device) is resorbable. In some embodiments, one or more components of system 10 comprise a resorbable polymer, and/or resorbable magnesium. In some embodiments, a component of system 10 that is configured to apply a force to tissue, and/or to stabilize another component of system 10 within tissue, comprises a resorbable material (e.g. such that the force and/or stabilization provided is temporary).

System 10 can be configured to identify tissue that has been treated (e.g. ablated), such as to properly treat adjacent tissue (e.g. provide a continuous treatment of a volume of tissue), and/or to avoid undesired treating of tissue that has already been treated. In some embodiments, algorithm 50 analyzes image data ID collected by system 10, and algorithm 50 records tissue that has been treated (e.g. ablated), such as by identifying and recording one or more anatomical landmarks in reference to tissue receiving energy (e.g. and recording the information as image data ID). Alternatively or additionally, algorithm 50 can analyze image data ID and identify tissue that has been treated by identifying characteristics of the treatment that can be found in the tissue after treatment (e.g. after ablation). In some embodiments, algorithm 50 is configured to perform an elastographic analysis and/or other tissue characterization analysis to identify treated tissue, or to position a marker of the treated tissue, as described immediately herebelow. In some embodiments, system 10 is configured to “mark” tissue (e.g. permanently and/or temporarily mark tissue), such as to provide one, two, or more markers used to identify tissue that has been ablated or otherwise treated by system 10 (e.g. one or more markers positioned within the treated tissue, on a periphery of treated tissue, and/or at a location proximate the treated tissue). EDM 150/250/350 can be configured to deliver energy (e.g. HIFU energy) at a particular level (e.g. a higher energy level than the remainder of the treated tissue) that causes an identifiable characteristic in the treated tissue (e.g. an energy delivery that causes cavitation within the treated tissue that can be later identified by system 10). In some embodiments, a clinician using device 100 may create one or more markers in the patient's tissue (e.g. proximate one or more tissue targets), and the clinician (e.g. manually) and/or algorithm 50 (e.g. in an automated or semi-automated fashion) can use the markers to complete a tissue treatment procedure based on the previously created markers.

In some embodiments, system 10 (e.g. via algorithm 50) can be configured to classify tissue, such as to differentiate treated tissue from untreated tissue, and/or to differentiate one type of tissue from another type of tissue. For example, system 10 can be configured to perform tissue elastography to classify tissue, such as when EDM 150/250/350 (e.g. configured to deliver ultrasound energy) and/or imaging device 910 (e.g. an MRI) deliver low frequency vibrations that are configured to measure the elasticity (e.g. stiffness) of tissue. In some embodiments, system 10 is configured to measure the elasticity of a portion of tissue in order to determine a level of ablation of that tissue (e.g. ablation delivered by system 10), and/or to determine if that tissue portion should be treated (e.g. treated due to its current level of elasticity). Alternatively or additionally, system 10 can be configured to track energy delivery (e.g. as correlated with image data ID) in order to track treated versus untreated tissue. Identifying and/or tracking of treated versus untreated tissue can be very important, such as to avoid undesired multiple treatments to a single tissue location and/or to prevent “missing” of treatment to a particular tissue location whose treatment is associated with improved efficacy of the entire procedure. The identifying and/or tracking of treated versus untreated tissue can be particularly important when a treatment procedure comprises treatment of a large number (e.g. at least 5, 10, 25, or 50) of different tissue targets via individual ablative energy deliveries (e.g. to treat a large number of targets within the patient's tongue or other airway tissue location). In some embodiments, tissue targets treated, and/or to be treated include anatomical locations (e.g. multiple tongue and/or other airway locations to be treated in a non-sequential manner) where tracking of treated versus untreated tissue is desired to prevent undesired multiple energy deliveries to the same tissue. As described herein, system 10 can be configured to create markers in tissue that can be used to track energy delivery, such as by delivering “marking energy” comprising energy (e.g. ultrasound energy) that is delivered at a different set of energy delivery settings than that used to simply ablate tissue (e.g. energy delivered at a higher level than the standard ablative energy, to cause detectable modifications to the tissue that is marked). In some embodiments, system 10 is configured to deliver marking energy in a particular pattern to mark tissue (e.g. two, three or more lines of HIFU or other energy delivery), such as a pattern that can be detected via algorithm 50 using a pattern recognition algorithm. System 10 can be configured to deliver marking energy along various locations of a periphery of a volume of tissue ablated (and/or to be ablated) by system 10, such as to create an identifiable (e.g. via algorithm 50) “treatment boundary”. In some embodiments, algorithm 50 is configured to identify particular tissue types, and/or combinations of tissue types (either or both “tissue types” herein), and to identify these particular tissue types in image data ID. Tissue type data (“data TTD”) can be used in planning a treatment procedure (e.g. defined as reference points) in which certain tissue types are to be avoided for treatment (e.g. used as a boundary for treatment), and/or when certain tissue types are to be treated (e.g. receive ablative energy). In some embodiments, tissue type data TTD comprises nerve location data, bone location data, blood vessel wall location data, and/or blood location data, such as when the identified tissue comprises tissue that are to be preserved (e.g. characterized as non-target tissue and not ablated). In some embodiments, system 10 (e.g. algorithm 50) is configured to provide a treatment plan using (e.g. based on an analysis of) two or more of: tissue type data TTD information; marked tissue information (e.g. tissue marked by EDM 150/250/350 as described herein, such as tissue identifiable by image analysis, elastography, and/or other analysis performed by algorithm 50); and/or clinician provided information (e.g. including clinician confirmation information). In some embodiments, algorithm 50 comprises an AI algorithm configured to perform an elastography analysis that differentiates treated from untreated tissue (e.g. and records the results in image data ID and/or registration data RD). In these embodiments, a treatment plan (e.g. as created by the clinician, system 10, or both) can safely include energy delivery to multiple targets, in a non-sequential manner, such as to avoid adversely affecting non-target tissue (e.g. the high temperatures that may result in sequentially treating multiple tissue targets in close proximity to one another). In some embodiments, algorithm 50 is configured to compensate for, and/or at least identify (e.g. and enter an alert mode), patient movement and/or undesired movement of EDM 150/250/350 relative to the patient.

System 10 can be configured to produce a temperature map of target tissue to be treated, such as a temperature map included in image data ID that includes both target tissue locations as well as non-target tissue locations that are proximate the target tissue locations. The temperature map produced by system 10 can be produced during delivery of energy (e.g. in real-time), such as when the temperature map is used to adjust energy delivery by EDM 150/250/350, and/or to identify zones of tissue that have been ablated (e.g. versus zones that have not, as described herein). In some embodiments, imaging device 910 comprises an MRI, and system 10 is configured (e.g. via algorithm 50) to produce a temperature map based on MR thermometry.

In some embodiments, system 10 is configured to deliver energy (e.g. ablative energy, marking energy, and/or other energy), to a first set of one or more particular volumes of target tissue using a first set of energy delivery settings, and to deliver energy (e.g. ablative energy, marking energy, and/or other energy) to a second set of one or more particular volumes of target tissue using a second set of energy delivery settings. The first and second sets of target tissue to be treated can include multiple discrete tissue targets, such as at least 5, 10, 25, and/or 50 tissue targets, and can include tissue types selected from the group consisting of: tissue of the tongue or other airway location (e.g. as ablated in a sleep apnea treatment procedure); hair follicle or other hair segment tissue (e.g. as ablated in a hair removal procedure); tumor tissue (e.g. as ablated in a cancer or other tumor treatment procedure); prostate tissue (e.g. as ablated in a BPH procedure); and/or brain tissue (e.g. as treated in an epileptic focus or other brain tissue treatment procedure). In these embodiments, there can be differences in the first and second energy delivery settings, such as differences that are configured to compensate for differences in the target tissue (e.g. different volumes of target tissue, types of tissue in the target tissue) and/or to avoid damage to particular non-target tissue proximate the target tissue (e.g. to avoid adversely affecting nerves, blood vessels, and other potential non-target tissue). The differences in the first and second energy delivery settings can include one or more differences in: type of energy delivered (e.g. ultrasound energy versus electromagnetic, light, chemical, and/or other energy form); amplitude of energy delivery; frequency of energy delivery; waveform of energy delivery (e.g. waveform shape); duty cycle of energy delivery; modulation of energy delivery; steering of energy delivery; focusing of energy delivery; and combinations of these. In some embodiments, system 10 delivers HIFU, focused ultrasound, and/or other ultrasound energy to a first volume of tissue at a first frequency, and to a second volume of tissue, smaller than the first volume of tissue, at a second frequency that is higher than the first frequency. The higher frequency can be used to selectively avoid adversely affecting non-target tissue near the target tissue, such as nerves and/or blood vessels that are near the target tissue to be ablated.

In some embodiments, all or a portion of EDD 100, and/or another component of system 10, is configured to be robotically manipulated by algorithm 50, such as when algorithm 50 comprises an AI algorithm configured to cause micromovements of EDM 150 during a tissue treatment and/or diagnostic procedure. In some embodiments, algorithm 50 is configured to cause EDM 150 to move based on an analysis of the anatomical location of non-target tissue (e.g. nerves) that are proximate to one or more tissue targets to be treated (e.g. to prevent damage to the non-target tissue). For example, during an ablative energy delivery, algorithm 50 can be configured to make small adjustments in EDM 150 position to avoid undesired tissue damage. In these embodiments, the identification of non-target tissue (e.g. as recorded in image data ID) can be: performed by a clinician; determined automatically by system 10 (e.g. via an AI-based algorithm 50); and/or identified via a combination of algorithm 50 identification and clinician confirmation. In some embodiments, system 10 is configured to stop energy delivery if an undesired state is detected by algorithm 50, such as when algorithm 50 determines that ablative energy is being delivered to non-target tissue, and/or any circumstance in which non-target tissue is being adversely affected.

In some embodiments, system 10 is configured to treat a blood vessel (e.g. one or more blood vessels supplying blood to target tissue) that are under a maximum diameter (e.g. vessels above the maximum are not treated), such as a maximum diameter of 2 mm, 1 mm, and/or 0.5 mm. In some embodiments, system 10 is configured to deliver energy (e.g. ultrasound energy) to treat a blood vessel, where the frequency of the energy delivered is based on the diameter of the segment of the blood vessel being treated, and/or the distance between the blood vessel segment and EDM 150 (e.g. the distance between the blood vessel segment and energy delivery elements 159).

System 10 can be configured to treat various medical conditions of a patient, such as one, two, or more medical conditions selected from the group consisting of: sleep apnea; presence of tumors and/or cysts; an ovarian condition; a cosmesis issue; epilepsy, cognitive impairment, and/or other neurological disorder; pain; a prostate issue (e.g. benign prostatic hyperplasia); a cardiac condition such as atrial fibrillation and/or other arrhythmia; a medical condition in which ablation of nerve tissue provides a therapeutic benefit; and combinations of these.

System 10 can be configured to allow one or more operators (e.g. a clinician, nurse, technician, and/or other health care provider of the patient) to perform a medical procedure on a patient. As described hereabove, system 10 can comprise one, two, three, or more energy delivery devices for use in a medical procedure, such as EDD 100, EDD 100′, and/or EDD 100″ shown (singly or collectively EDD 100). Similarly, system 10 can comprise one, two, three, or more chronic energy delivery devices, such as CEDD 200, CEDD 200′, and/or CEDD 200″ shown (singly or collectively CEDD 200), one, two, three or more force-applying devices, such as FAD 300, FAD 300′, and/or FAD 300″ shown (singly or collectively FAD 300), and/or one, two, or more treatment devices 930. In some embodiments, system 10 comprises multiples of devices 100, 200, 300, and/or 930 that are used in a single clinical procedure to diagnose and/or treat a patient with an undesired medical condition (e.g. a disease or disorder). In some embodiments, system 10 comprises multiples of devices 100, 200, 300, and/or 930 that are used in two or more separate clinical procedures (e.g. performed on separate days) to treat a medical condition of a patient. For example, multiple devices 100, 200, 300, and/or 930 can be used in one, two, or more clinical procedures performed to treat sleep apnea. The one, two or more clinical procedures can include one, two, or more tissue treatments selected from the group consisting of: tissue stimulation (e.g. as performed by EDD 100 and/or CEDD 200); tissue ablation (e.g. as performed by EDD 100); tissue debulking (e.g. as performed by EDD 100); muscle strengthening (e.g. as performed by EDD 100, CEDD 200, and/or FAD 300); tissue scaffolding (e.g. as scaffolded by one or more forces applied by FAD 300 and/or by a treatment device 930), a treatment provided by treatment device 930; and combinations of these, each as described herein.

System 10 can be configured to treat target tissue located in various anatomical locations of a patient, such as when target tissue comprises one, two, or more tissue types selected from the group consisting of: airway tissue; bone (e.g. facial bone); cartilage; tumor tissue; hair segment tissue (e.g. all or a portion of a hair shaft, hair root, hair follicle, hair bulb, and/or a segment of a blood vessel providing blood to tissue of a hair bulb); heart tissue; brain tissue; liver tissue; kidney tissue; pancreatic tissue; organ tissue; blood; and combinations of these. In some embodiments, target tissue comprises one, two, or more types of “airway tissue”, such as one or more tissues (e.g. muscle, fat, and/or nerve tissue) selected from the group consisting of: adenoid tissue (e.g. also referred to as pharyngeal tonsil or nasopharyngeal tonsil tissue); cartilage tissue proximate the airway; epiglottis tissue; facial bone proximate the airway; genioglossus tissue; geniohyoid tissue (e.g. the C1 branch of the geniohyoid muscle); glossopharyngeal tissue; hypoglossal tissue; lymphoid tissue proximate the airway; nasal septum tissue; palatoglossus tissue; pharyngeal wall tissue (e.g. lateral pharyngeal wall tissue); soft palate tissue; stylopharyngeus tissue; tensor veli palatini tissue; tongue tissue (e.g. including adipocytes and other fat of the tongue, as well as the intrinsic or extrinsic muscles of the tongue); tonsil tissue (e.g. palatine tonsil and/or lingual tonsil tissue); turbinate tissue (e.g. inferior turbinate tissue); vagus nerve tissue; and combinations of these.

EDD 100 and/or CEDD 200 can be configured to deliver energy in a closed-loop mode (i.e. a closed-loop mode of energy delivery and/or other closed-loop mode of operation), such as when one or more sensors of system 10 (e.g. a sensor based functional element 199, 299, 399, 599, and/or 999), provide patient and/or system information that is used to adjust energy being delivered by device 100/200. Energy delivery by EDM 150/250 can be adjusted in a closed-loop mode based on a system 10 parameter and/or based on a patient parameter (e.g. a patient physiologic parameter and/or a patient environment parameter, each as described herein). Energy delivery by EDM 150/250 can be adjusted based on image data ID described herein, such as to redirect energy delivery (e.g. due to detected patient motion and/or undesired EDM 150/250 motion) and/or to change one or more energy delivery settings (e.g. due to an ablation level or other treatment level as determined by algorithm 50 using image data ID). In some embodiments, image data ID is used to determine when a treatment (e.g. an ablation) is sufficient, such as when algorithm 50 analyzes ultrasound-based image data to confirm sufficient change in tissue characteristics have occurred. In some embodiments, image data ID comprises blood flow data (e.g. obtained via Doppler ultrasound), and energy delivery for treatment is based on levels and/or changes of flow of blood comprising target tissue and/or non-target tissue.

FAD 300 can be configured to apply force in a closed-loop mode (i.e. a closed-loop mode of applying a force and/or other closed-loop mode of operation), such as when one or more sensors of system 10 (e.g. a sensor based functional element 199, 299, 399, 599, and/or 999), provide patient and/or system information that is used to adjust energy being delivered by FAD 300. Force applied by FAD 300 can be adjusted in a closed-loop mode based on a system 10 parameter and/or based on a patient parameter (e.g. a patient physiologic parameter and/or a patient environment parameter, each as described herein). Force applied by FAD 300 can be adjusted in a force titration procedure, as described herein. Force applied by FAD 300 can be adjusted intermittently, and/or continuously. Force applied by FAD 300 can be adjusted based on time of day, and/or a patient physiologic parameter (e.g. a sleep parameter such as snoring). Force applied by FAD 300 can be adjusted to compensate for patient breathing (e.g. as determined via signals provided by a respiration sensor of system 10). Force applied by FAD 300 can be adjusted based on image data ID described herein, such as to adjust force applied due to detected patient motion and/or undesired FAD 300 motion (e.g. as determined by algorithm 50 using image data ID). In some embodiments, image data ID is used to determine when an applied force has reached a sufficient level (e.g. a sufficient level of force and/or a sufficient duration of time in which force has been applied), such as when algorithm 50 analyzes ultrasound-based image data to confirm sufficient change in tissue characteristics have occurred.

In some embodiments, energy delivery by one or more components of system 10 (e.g. devices 100, 200, and/or 300) is configured to be manually activated (e.g. “turned on” or simply enabled to start energy and/or force delivery, and/or “turned off” or disabled from delivering energy and/or force) by an operator, such as when system 10 is activated by the patient prior to the patient going to sleep and/or by a clinician of the patient when it is determined that therapeutic energy delivery should be started. Alternatively or additionally, system 10 can be configured to be activated automatically. For example, system 10 can initiate or otherwise enable energy delivery (e.g. by CEDD 200) and/or force delivery (e.g. by FAD 300) at a certain time of day (e.g. a time of night when the patient normally goes to sleep) and/or when an assessment of a patient physiologic parameter indicates energy should be delivered (e.g. when system 10 determines that the patient is sleeping and/or snoring, such as via a sensor and algorithm 50). Alternatively, system 10 can stop energy and/or force delivery automatically, such as at a certain time of day and/or based on a patient physiologic parameter (e.g. algorithm 50 determines the patient is awake, has stopped snoring, and/or otherwise should not receive energy delivery). In some embodiments, system 10 is configured to deliver energy (e.g. by CEDD 200) and/or apply a force (e.g. by FAD 300) during a certain patient activity, such as talking and/or singing, such as to exercise the muscles used to talk and/or sing.

System 10 can be operated in a closed-loop mode in which modification of energy delivery (e.g. modify frequency, amplitude, wave shape, selection of energy delivery elements delivering energy, and/or other energy delivery parameter) is performed if an undesired energy delivery state is encountered. Undesired energy delivery states include but are not limited to: temperature of tissue and/or temperature of a portion of a system 10 component out of a desired temperature range (e.g. above a maximum temperature and/or below a minimum temperature), amount of tissue ablated per unit time at an unacceptable rate; amount of time delivering energy (e.g. to a single location) above a threshold; tissue state and/or change of state (e.g. elasticity state) at an undesired level; and combinations of these.

FAD 300 can be configured to operate in a closed-loop mode, such as when one or more sensors of system 10, as described herein, provide patient and/or system information that is used to adjust FAD 300, such as to adjust the force applied by FAD 300 to target tissue. In some embodiments, system 10 is configured to detect and/or predict an undesired sleep condition such as snoring and/or an apnea event, and system 10 (e.g. via algorithm 50) is configured to adjust FAD 300 to stop, limit, and/or prevent the undesired sleep condition. As described hereabove, FAD 300 can include EDM 350, and EDM 350 can be configured to operate in a closed-loop mode of energy delivery, similar to that described in reference to EDM 150 and EDM 250 hereabove.

In some embodiments, energy delivery by EDD 100 and/or CEDD 200 while performing a tissue reduction procedure is provided in a closed-loop mode based on a measurement (e.g. by one, two, or more sensors of system 10) of a parameter selected from the group consisting of: temperature such as tissue temperature and/or temperature of a portion of a component of system 10 (e.g. a portion of EDD 100 and/or CEDD 200); patient motion and/or patient position (e.g. where source and/or direction of energy delivery is modified to compensate for patient motion); a patient physiologic parameter; a patient environment parameter; a system 10 parameter; and combinations of these. In some embodiments, energy delivery by EDD 100 and/or CEDD 200 is delivered in a closed-loop mode that is based on a tissue ablation analysis, as described herein. For example, system 10 can be configured to stop energy delivery when a sufficient level of tissue ablation is achieved (e.g. as recognized by algorithm 50, such as by using image data ID collected by one or more components of system 10, such as EDM 150 and/or EDM 250 as described herein).

In some embodiments, energy delivery by EDD 100 and/or CEDD 200 while performing a tissue enhancement procedure is provided in a closed-loop mode based on a measurement (e.g. by one, two, or more sensors of system 10) of a parameter selected from the group consisting of: temperature such as tissue temperature and/or temperature of a portion of a component of system 10 (e.g. EDD 100 and/or CEDD 200); patient motion (e.g. where direction of energy delivery is modified to compensate for patient motion); a patient physiologic parameter such as blood pressure, heart rate, and/or respiration rate; a patient sleep parameter (e.g. such as snoring level, patient body position during sleep, and/or other sleep parameter as described herein); a system 10 parameter; and combinations of these.

EDD 100 can be configured, as described herein, to both image tissue as well as perform a tissue reduction or other tissue treatment procedure (e.g. a tissue treatment procedure performed on at least a portion of the tissue that has been imaged). Image data ID produced during imaging can be used by an operator (e.g. manually) and/or automatically by system 10 (e.g. via algorithm 50), to differentiate target tissue to be treated and non-target tissue in which to avoid adverse effects (e.g. avoid delivery of sufficient energy to cause necrosis or other volume reducing effect). For example, the target tissue to receive a tissue reduction procedure can comprise fat tissue (e.g. fat tissue of the tongue or other airway tissue), and the non-target tissue to avoid being damaged can comprise blood vessel tissue, nerve tissue, and/or muscle tissue (e.g. non-target tissue proximate the fat tissue being reduced and/or otherwise treated). Using image data ID, a treatment plan can be generated (e.g. by system 10 via an AI-based or other form of algorithm 50) that delivers a pattern of energy (e.g. relatively short bursts of ultrasound energy) that liquefies fat cells of the target tissue while avoiding damage to neighboring non-target tissue. In some embodiments, both fat tissue and a small portion of muscle tissue is treated (e.g. such as to cause coagulative necrosis of the muscle tissue), such as when the additional treatment to the muscle tissue provides an enhanced therapeutic benefit to the patient (e.g. a sleep apnea patient). In some embodiments, non-target tissue comprises blood vessel tissue, nerve tissue, and/or mucosal tissue (e.g. mucosal tissue of an airway), and system 10 is configured to analyze image data ID to develop a treatment plan that avoids damage to those types of non-target tissue, while ablating the intended target tissue (e.g. fat and/or muscle tissue).

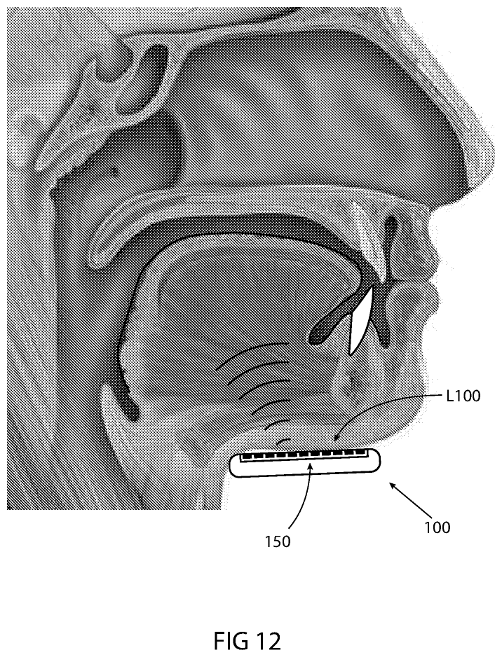

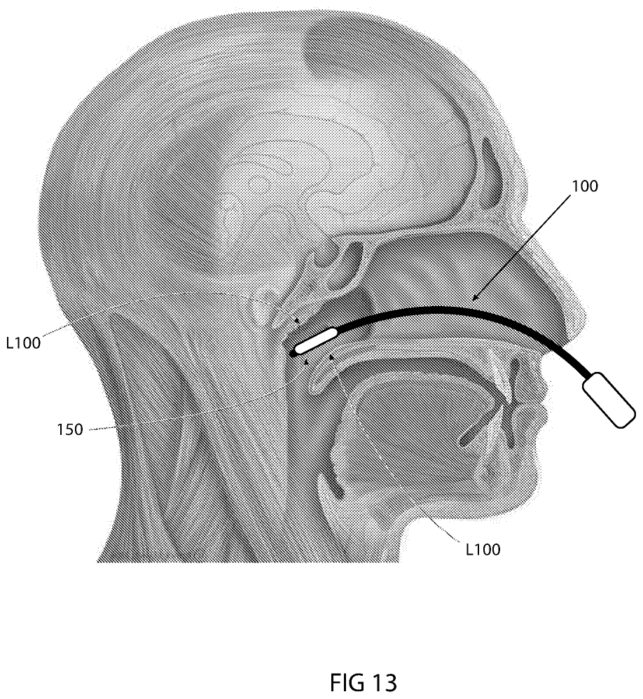

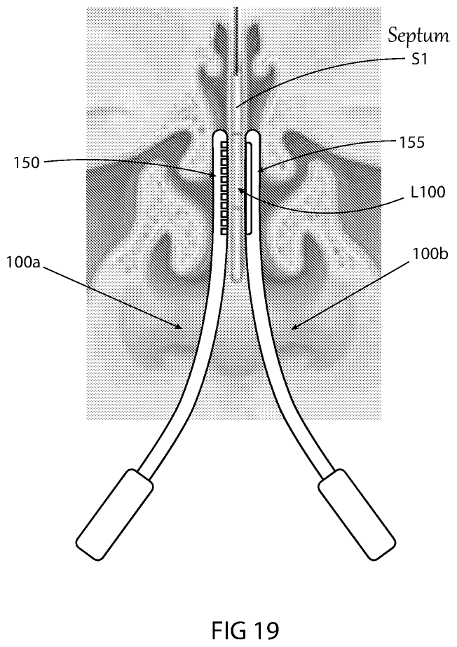

EDM 150 of EDD 100 can be configured to be positioned at a location L100 that is accessed via the patient's mouth (e.g. as shown in FIG. 18B) and/or nose (e.g. as shown in FIGS. 13 and 19). For example, EDM 150 (e.g. comprising an array of ultrasound transducers) can be advanced through the mouth and/or a nostril of the patient such that a tissue treatment (e.g. an ablation and/or stimulation treatment) can be performed on the patient's tongue, soft palate, tonsil, muscle tissue of the airway, and/or other airway tissue. Alternatively or additionally, EDM 150 can be positioned on the skin of the patient, such as when positioned on the skin located under the patient's chin (e.g. as shown in FIG. 12), such as to deliver treatment energy to tissue of the tongue and/or genioglossus (e.g. without the need of the patient's mouth being open during the procedure).

In some embodiments, system 10 is configured to perform a medical procedure to treat a sleep apnea condition of the patient, such as when target tissue comprises airway tissue as defined herein. In these embodiments, target tissue can be treated using a tissue reduction procedure (e.g. a procedure in which airway tissue is ablated using EDD 100 and/or CEDD 200), a tissue enhancement procedure (e.g. a procedure in which airway tissue is stimulated using EDD 100 and/or CEDD 200), and/or a procedure in which force is applied to tissue (e.g. using FAD 300). In some embodiments, at least two of EDD 100, CEDD 200, or FAD 300 are used, in a single clinical procedure or multiple clinical procedures, to treat a sleep apnea patient. In some embodiments, at least one EDD 100, at least one CEDD 200, and at least one FAD 300 are used, in a single clinical procedure or multiple clinical procedures, to treat a sleep apnea patent.

As described herein, system 10 can be configured to perform a series of clinical procedures on a patient, such as a sleep apnea patient. In some embodiments, system 10 is configured to be used to: perform a first procedure in which the volume of at least tonsil tissue is reduced (e.g. volume reduced by ablation or other tissue reduction procedure using EDD 100 as described herein); and perform a second procedure (e.g. subsequent to the first procedure) in which muscle strengthening is performed (e.g. muscle strengthening performed by delivery of stimulation energy or other tissue enhancement procedure as described herein). In some embodiments, system 10 is configured to be used to: perform a first procedure in which a jaw expansion procedure is performed (e.g. a surgical procedure and/or a procedure in which bone is removed and/or weakened using energy delivered by EDD 100); and perform a second procedure (e.g. subsequent to the first procedure) in which system 10 is used to ablate airway tissue (e.g. tongue, tonsil, and/or other airway tissue is ablated using EDD 100), stimulate airway tissue (e.g. via CEDD 200), and/or apply a force to airway tissue (e.g. via FAD 300). In some embodiments, system 10 is configured to be used to: perform a first procedure in which the volume of at least the patient's tongue is reduced (e.g. via ablation and/or other energy delivery by EDD 100); and perform a second procedure (e.g. subsequent to the first procedure) in which force is applied to at least the patient's tongue (e.g. via FAD 300). In some embodiments, system 10 is configured to be used to: perform a first procedure in which the volume of airway tissue is reduced (e.g. tongue, tonsil, and/or other airway tissue is reduced using EDD 100); perform a second procedure (e.g. subsequent to the first procedure) in which tissue of the upper airway is toned or otherwise enhanced (e.g. toning of upper airway muscles associated with the top jaw or other enhancement performed using EDD 100); and perform a third procedure (e.g. subsequent to the first procedure and/or the second procedure) in which stimulation energy is delivered and/or force is applied to airway tissue (e.g. via CEDD 200 and/or FAD 300, respectively). In some embodiments, system 10 is configured to be used to: perform a first procedure in which the volume of at least tonsil tissue is reduced (e.g. volume reduced by ablation or other tissue reduction procedure using EDD 100 as described herein); and perform a second procedure (e.g. subsequent to the first) in which force is applied to airway tissue (e.g. via FAD 300).

In some embodiments, system 10 is configured to treat a sleep apnea patient using a treatment device 930, such as a treatment device comprising a CPAP and/or an oral appliance. In these embodiments, system 10 can utilize one or more of EDD 100, CEDD 200, and/or FAD 300 to improve the efficacy of the treatment device 930. For example, a patient that is using CPAP at a first level (e.g. a first pressure level or air-flow level), can receive a treatment using system 10 (e.g. an airway tissue ablation or other procedure as described herein), after which the patient can: avoid the use of CPAP (e.g. and still avoid significant adverse effects of sleep apnea), or use their CPAP at a second level that is reduced from the first level (e.g. at a pressure level and/or air-flow level that is reduced from the level used prior to the treatment using system 10). In some embodiments, a patient is taking one or more pharmaceutical medications prior to a treatment performed by system 10, and the patient's use of the pharmaceutical agents is reduced or avoided after one or more treatments are performed, as described herein, using system 10.

EDD 100, via energy delivered by EDM 150 (e.g. HIFU or other ultrasound energy), can be used to perform a tissue reduction procedure on airway tissue of a patient, such as tonsil tissue of the patient (e.g. a sleep apnea patient). In some embodiments, system 10 is configured to reduce a significant portion of tissue volume of a tonsil, such as by removing at least 50% of the pre-treated tonsil volume, but not more than 90 or 95% of the pre-treated volume. In other embodiments, system 10 is used to reduce the volume of a tonsil while leaving at least 20%, 30%, 40%, 50%, and/or 80% of the pre-treated volume of the tonsil, while still providing an improvement in a medical condition of the patient (e.g. an improvement in sleep apnea that may occur due to shrinkage of the tonsil tissue that occurs over time, as described herein). In these embodiments, actual volume of tonsil reduction can occur over time, such as time periods of at least 1 day, at least one week, and/or no more than 1 month. In some embodiments, all of the tissue treated (e.g. reduced), comprises subsurface tissue of the tonsil, in other words the surface tissue of the tonsil is left untreated, and the treatment (e.g. ablation and/or liquefication) of the subsurface tissue results in an overall volume reduction of the tonsil. In these embodiments, EDD 100 can include a cooling element (e.g. functional element 199 and/or spacer 251 comprises a cooling element) such that subsurface tissue can be ablated, while the surface tissue (e.g. being cooled prior to, during, and/or after delivery of energy) is not adversely affected. In some embodiments, the tonsil is treated using a EDM 150 including a capture portion, in which the tonsil is drawn into and/or otherwise positioned within the capture portion (e.g. as described in reference to FIGS. 9, 10, and/or 11 herein). In some embodiments, vacuum (e.g. as provided by console 500 and/or functional element 199 of EDD 100) is applied to the capture portion of EDM 150 to draw tissue to be treated into the capture portion. In some embodiments, target tissue to be treated comprises tonsil tissue that is drawn into (e.g. via vacuum) a capture portion of EDM 150, the movement of the tissue providing a separation of the tonsil tissue from muscle tissue. In other embodiments, target tissue comprising tonsil tissue is left in place (e.g. not drawn away from non-target tissue), and ablation of non-target tissue (e.g. muscle tissue proximate the target tonsil tissue) is avoided through precision of energy delivery (e.g. delivery of HIFU that is performed while simultaneously imaging the target and non-target tissue).

In some embodiments, EDD 100 is configured to reduce the volume of airway tissue that is positioned proximate muscle tissue that is not to be adversely affected (e.g. tonsil tissue to undergo volume reduction that is positioned in front of muscle tissue that is not to be ablated). In these embodiments, EDD 100 can comprise an array of ultrasound transducers (e.g. CMUT and/or piezo transducers) that deliver ultrasound energy (e.g. planar wave ultrasound energy) at a target frequency (e.g. approximately 6 MHz) that is configured to ablate a particular thickness of tissue (e.g. a thickness of 8-10 mm), while avoiding adversely affecting deeper tissue. In some embodiments, target tissue comprises a particular thickness, and non-target tissue is positioned behind the target tissue. In these embodiments, EDD 100 can be configured to ablate an inner portion of the thickness of the target tissue (e.g. no more than 80% of the full thickness), such as to avoid adversely affecting the non-target tissue (e.g. muscle tissue and/or nerve tissue) beneath the target tissue to be treated (e.g. volume reduced). In some embodiments, EDD 100 is configured to ablate target tissue (e.g. tonsil tissue) while avoiding damage to non-target tissue (e.g. the muscle bed beneath the tonsils) using image data ID (e.g. real time image data) collected by EDD 100. In these embodiments, EDD 100 can comprise an array of ultrasound transducers (e.g. CMUT and/or piezo transducers) that deliver focused ultrasound energy (e.g. HIFU) to target tissue, while avoiding undesired temperature increases in non-target tissue (e.g. using geometric information in the image data ID and/or tissue temperature information in the image data ID).

EDD 100 can be used by an operator to reduce a volume of fat tissue as described herein, such as fat tissue proximate the airway of the patient that is contributing to sleep apnea of the patient. For example, fat tissue of the tongue, tonsil, and/or luminal walls of an airway can be reduced via energy delivered via EDM 150. Fat tissue treated can comprise fat in the posterior of the tongue, fat in the walls of the patient's airway (e.g. along the lateral pharyngeal wall), and/or fat in the soft palate. In some embodiments, the energy delivered by EDM 150 (e.g. ultrasound energy) can be configured to liquefy fat tissue, as described herein, such as by delivering energy in a manner that avoids ablating tissue (e.g. avoids significant times at high temperatures, such as by avoiding long exposures to temperatures above 43° C., or any exposures above 60° C.).

System 10 can be configured to modify the shape of a segment of the airway of the patient's nasal cavity, such as via delivery of energy by EDD 100 and/or CEDD 200. For example, EDM 150 of EDD 100 can be positioned at a location within and/or proximate the surface of nasal cavity tissue, such as to perform a treatment procedure on the nasal cavity tissue (e.g. as described in reference to FIGS. 13 and 19 herein). Energy delivered by EDM 150 (e.g. HIFU and/or other ultrasound energy) can be delivered to create holes in the nasal septum, soften the nasal septum, or both, such that the nasal septum can be reshaped (e.g. over time) to increase the volume of the patient's airway proximate the nasal septum. In some embodiments, EDM 150 is configured to perform a modification of tissue selected from the group consisting of: create one or more holes in the nasal septum; thin or otherwise reduce the volume of cartilage proximate the nasal cavity; soften and/or otherwise reduce the rigidity of bone and/or cartilage proximate the nasal passageway; and combinations of these. In some embodiments, EDM 150 is configured to deliver energy to create holes and/or soften the nasal septum, such as to reshape and/or otherwise facilitate the reshaping (“reshape” herein) the nasal passageway, such as to improve a sleep apnea condition of the patient. In some embodiments, after the treatment energy is delivered by EDM 150, a treatment device 930 comprising an expandable structure (e.g. a balloon filled with room temperature and/or heated fluid, and/or another structure that can be expanded and/or deliver heat) is inserted proximate the treated segment of the airway, in order to reshape that portion and maintain the new shape. In some embodiments, after the treatment energy is delivered by EDM 150, a treatment device 930 comprising one or more stents (e.g. temporary stents) is positioned in and/or otherwise proximate the treated segment of the airway, in order to reshape that portion and maintain the new shape. In these embodiments, the stent-based treatment devices 930 can be removed after a period of no more than 3 months, 1 month, and/or one week. In some embodiments, energy delivery is performed by a set of devices positioned on either side of the nasal septum, such as when EDM 150a is positioned on one side of the septum and mirror 155 is positioned on the other side of the septum (e.g. a described in reference to FIG. 19 herein), or when a first EDM 150a and a second EDM 150b are positioned on opposite sides of the septum, such that either or both can deliver energy (e.g. ultrasound energy) to the septum.

In some embodiments, at least FAD 300 is used to treat a sleep apnea patient (e.g. such as when also EDD 100 and/or CEDD 200 are used to treat the patient). FAD 300 can be positioned proximate airway tissue, such that FAA 360 can scaffold the airway (e.g. move tissue out of the airway) and/or FAA 360 can tone, strengthen, and/or otherwise enhance the airway tissue (e.g. muscle tissue of the airway). In some embodiments, FAD 300 is attached to the pterygoid hamulus, such that when activated, FAA 360 can rotate to tension muscle tissue of the airway (e.g. to treat soft palate-based sleep apnea). FAA 360 can be positioned to apply a force to tissue of the tensor veli palatini, such as to move that tissue in a back and forth motion. In some embodiments, FAA 360 is positioned to apply force to tissue, in one, two, three, or more directions. In some embodiments, FAA 360 is positioned to apply force to one, two, three, or more tissues selected from the group consisting of: tensor veli palatini tissue; levator veli palatini tissue; genioglossus tissue (e.g. to cause the tongue to pull forward); internal nasal valve tissue; tongue tissue (e.g. to cause contraction when the force is applied); and combinations of these.

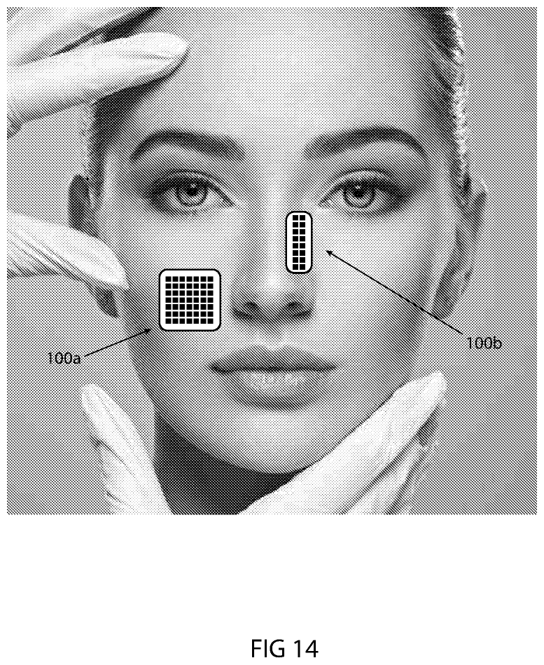

System 10 can be configured to perform transfacial energy delivery, such as via delivery of energy by EDD 100 and/or CEDD 200 through the patient's skin surface to tissue beneath the skin, such as is described in reference to FIG. 14 herein. In these embodiments, system 10 can be configured to deliver energy to muscle, fat, and/or bone in the face of the patient. In some embodiments, EDM 150 of EDD 100 delivers energy to reduce the volume of and/or at least weaken bone tissue, such as to subsequently perform a distraction osteogenesis maxillary expansion (DOME) procedure on a sleep apnea patient.

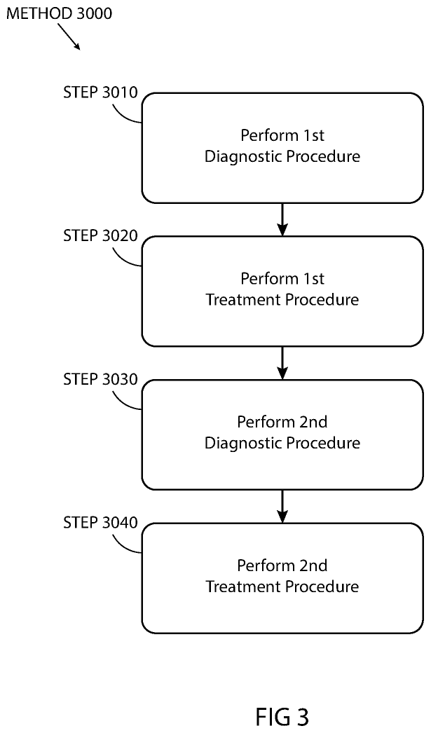

System 10 can be configured to perform a treatment on a patient (e.g. a sleep apnea patient) that includes the performance of multiple, sequential treatment plans, such as a sequence of treatment plans that each may use one, two or more components of system 10 (e.g. one, two or more of devices 100, 200, and/or 300) that are used to perform one or more diagnostic procedures, and/or one or more therapeutic procedures. In some embodiments, system 10 is configured to perform a treatment plan as described in reference to FIG. 3 herein. Performance of an “initial treatment plan” performed using system 10, can be configured based on current physiologic state (e.g. current sleep apnea conditions) of the patient, as well as any previous treatments performed (e.g. using system 10 or otherwise). Each “subsequent treatment plan”, can also be based on the current physiologic state, as well as all previous treatments performed.

In some embodiments, an initial treatment plan can include using system 10 to perform one or more tissue reduction procedures, one or more tissue enhancement procedures, or at least one tissue reduction procedure and at least one tissue enhancement procedure. For example, an initial treatment plan can include one, two, or more of: a tissue ablation procedure performed on soft tissue, such as adenoid, palatine tonsil, lingual tonsil, inferior turbinate, and/or lingual fat tissue; a tissue deformation procedure performed on hard tissue, such as an anterior septoplasty procedure performed using EDD 100 or otherwise, and/or a deformation procedure (e.g. distraction procedure) performed on the maxilla (e.g. the piriform rims and/or lateral buttress of the maxilla) using EDD 100 or otherwise; and a muscle strengthening procedure such as a strengthening procedure performed on muscles of the tongue (e.g. intrinsic muscles of the tongue) and/or an uvulopalatopharyngoplasty (UPPP) performed on upper airway tissue (e.g. tensor veli palatini, levator palatini, palatopharyngeus, palatoglossus, genioglossus, and/or geniohyoid tissue).

A second treatment plan (e.g. a plan that is determined after the first treatment plan described immediately hereabove is performed) can comprise a treatment plan comprising a tissue enhancement procedure (e.g. as performed using a device 100/200/300). For example, the second treatment plan can comprise a muscle strengthening procedure, such as a neurostimulation procedure (e.g. via energy delivery from CEDD 200) in which the trigeminal nerve (the fifth cranial nerve, CN V), hypoglossal nerve (the twelfth paired cranial nerve, CN XII), and/or vagus nerve (the tenth cranial nerve, CN X) are stimulated (e.g. unilaterally and/or bilaterally, such as via an intraoral and/or cervical approach). Alternatively or additionally, the muscle strengthening procedure of the second treatment plan can be performed by applying force to one or more muscles (e.g. those associated with the nerves described immediately hereabove), such as via one or more forces applied by FAD 300 as described herein.

A third treatment plan (e.g. a plan that is determined after the second treatment plan described immediately hereabove is performed), can comprise a tissue stabilization and/or expansion procedure. In some embodiments, an FAD 300 or treatment device 930 (e.g. comprising a vibration-reducing implant) can be positioned in or at least proximate the soft palate or other airway location. In some embodiments, the third treatment plan comprises positioning an FAD 300 in and/or otherwise proximate airway tissue, such as to scaffold the airway, strengthen and/or otherwise enhance the muscle tissue of the airway, and/or provide another therapeutic benefit. In some embodiments, all or a portion of the implanted FAD 300 is configured to remain in the patient for at least 1 week, 1 month, and/or 3 months. In some embodiments, all or a portion of the implanted FAD 300 is configured to be resorbed, such as a component that is configured to resorb after at least 1 week, 1 month, and/or 3 months.

In some embodiments, the first, second, and/or third treatment plans described immediately hereabove are performed in any order. In some embodiments, one of the first, second, or third treatment plans is not performed on the patient.

In some embodiments, system 10 comprises a functional element 999 that comprises a sensor that is implanted in the patient and configured to detect patient breathing, such as a sensor positioned proximate the lungs; the external and intercostal muscles (e.g. between the external and intercostal muscles); and/or other location under the patient's skin. In some embodiments, functional element 999 comprises a sensor that is positioned external to the patient, such as on or otherwise proximate the patient's skin, and is configured to detect patient breathing. In these embodiments, a CEDD 200 and/or an FAD 300 can be configured in a closed-loop mode that, via algorithm 50 as described herein, adjusts energy and/or force delivery, respectively, based on the patient's breathing. For example, CEDD 200 via EDM 250 can be configured to deliver adjustable energy to the hypoglossal nerve (e.g. to produce tongue protrusion).

In some embodiments, EDD 100 and/or CEDD 200 is configured to deliver ultrasound energy, as described herein, to interact with a pharmaceutical drug (e.g. agent 920) that has been delivered to the patient. The interaction can comprise a release (e.g. from a carrier) and/or other activation of the drug, and/or the interaction can comprise an enhancement of the effects of the drug. In some embodiments, the drug comprises one or more visualizable components (e.g. radiographic and/or ultrasonically reflective components) that system 10 uses to identify the location of the drug, and then precisely deliver the ultrasound energy to cause the interaction.

In some embodiments, EDD 100 and/or CEDD 200 is configured to deliver energy to treat a cyst of the patient. In some embodiments, energy is delivered to a relatively small cyst, such as to image and/or treat the cyst (e.g. ablate the entire cyst or at least reduce the volume of the cyst). In some embodiments, a larger cyst is to be treated, and the fluid from the cyst is drained (e.g. via a treatment device 930), after which energy is delivered to image and/or treat the cyst.

In some embodiments, FAD 300 comprises at least a portion (e.g. FAA 360) that is implanted in the patient at an implant location (e.g. L300 described herein) that is chosen to treat subglottic stenosis (e.g. a stenosis that results as a complication of previous intubation of the patient). In these embodiments, FAA 360 can be anchored to laryngeal cartilage such that one or more actuators of FAA 360 apply force to the stenosis (e.g. scaffold the stenosis).

FAD 300 can comprise at least a portion (e.g. FAA 360) that is implanted in the patient at an implant location (e.g. L300 described herein) that is chosen to treat pelvic organ prolapse, and/or urinary incontinence. One or more actuators of FAA 360 can be configured to apply force to one or more tissue locations (e.g. one or more volumes of target tissue) to treat pelvic organ prolapse and/or to treat urinary incontinence. FAA 360 can be normally configured (e.g. resiliently biased) in a deployed state (e.g. minimal or no energy is consumed while applying force to tissue, and energy is applied to transition to the undeployed state). Alternatively or additionally, FAA 360 can be normally configured (e.g. resiliently biased) in an undeployed state (e.g. energy is applied to FAA 360 to transition to the deployed state). In some embodiments, one or more actuators of FAA 360 can be positioned to apply force to tissue (e.g. urethral tissue) to prevent undesired leakage of urine. In these embodiments, FAA 360 can be normally configured (e.g. resiliently biased or otherwise configured) in a deployed state (e.g. actuators extended to apply force to tissue), and the patient can (via an externally placed user interface 390 and/or other user interface as described herein) cause FAA 360 to transition to the undeployed state (e.g. retract the one or more actuators of FAA 360 to stop applying force to tissue), such as to allow urination.

System 10 can be configured to provide a therapy to the lungs of a patient, such as to improve flow of air through one or both lungs of the patient. In some embodiments, system 10 is configured to measure positive end-expiratory pressure (PEEP) prior to, during, and/or after treatment of the patient's lungs. In these embodiments, PEEP can be measured via an EDD 100 and/or an imaging device 910 comprising an ultrasound imager. For example, EDM 150 can comprise an array of ultrasound transducers configured to produce image data ID from which PEEP measurements can be determined (e.g. via algorithm 50). The PEEP measurements can be used to create a treatment plan, as defined herein, such as to set and/or modify (“set” herein) one or more energy delivery settings of EDM 150, such as one or more energy delivery settings related to a tissue reduction procedure (e.g. a tissue ablation procedure) and/or a tissue enhancement procedure (e.g. a tissue stimulation procedure). Alternatively or additionally the PEEP measurement can be used to perform a prognosis of the treatment by system 10 (e.g. algorithm 50 can predict an outcome of a procedure prior to performing the procedure, during the performance of the procedure, and/or after the procedure has been completed).

Referring now to FIG. 2, a flow chart of a method of providing a treatment in a closed-loop arrangement is illustrated, consistent with the present inventive concepts. Method 2000 comprises multiple steps that can be performed automatically and/or semi-automatically (“automatically” herein) to deliver energy (e.g. stimulation energy) and/or otherwise provide a treatment in a form that varies based on one or more measured patient physiologic parameters, patient environment parameters, and/or other parameters. Method 2000 of FIG. 2 is described using system 10 of the present inventive concepts and its components.

In STEP 2010, EDD 100 and/or CEDD 200 (singly or collectively “device 100/200” herein) has been positioned proximate target tissue (e.g. one or more volumes of patient tissue), and device 100/200 is configured to deliver energy to the target tissue (e.g. to stimulate and/or otherwise treat the target tissue). Device 100/200 can be implanted within patient tissue, and/or positioned on a tissue surface (e.g. the skin of the patient and/or the inner lining of an airway of the patient). In STEP 2010, device 100/200 can be delivering energy to the patient, or can be in a “standby” mode in which no energy is being delivered.

In STEP 2020, device 100/200 and/or another component of system 10 monitors one or more parameters of the patient. The patient parameters can be recorded by a sensor-based functional element, such as functional element 199, 299, 599, and/or 999 described herein. In some embodiments, the patient parameter comprises one, two, or more parameters selected from the group consisting of: blood pressure; heart rate; respiration rate; a snoring parameter (e.g. snoring level); perspiration; patient position (e.g. on their back, side, and/or stomach when sleeping); blood gas level; blood glucose level; and combinations of these. In some embodiments, the patient parameters comprise parameters that can be recorded (e.g. measured by one, two, or more sensors) and/or provided by a patient's wearable device, such as a smart watch, an activity-recording device (e.g. worn on the patient's wrist), and/or other portable device. In some embodiments, a patient's respiration rate is measured by a sensor (e.g. a sensor-based functional element 299, 399, and/or 999) that comprises a wired or wireless sensor.

In STEP 2030, the one or more patient parameters recorded by system 10 can be analyzed (e.g. by algorithm 50) such as to determine if a threshold has been exceeded. The threshold can comprise a minimum or maximum level for one or more patient parameters. Alternatively or additionally, the threshold can be related to a mathematical combination of two or more patient parameters. The thresholds used in STEP 2030 can be related to a level of one, two, or more patient parameters that correlate to ineffective or otherwise undesired level of stimulation or other energy currently being delivered by device 100/200 (e.g. an energy delivery level that should be changed or stopped).

If the threshold is not exceeded, method 2000 continues with the monitoring of patient parameters of STEP 2020 (e.g. without modifying the current energy delivery or other treatment settings). If the threshold is exceeded, method 2000 continues with STEP 2040.

In STEP 2040, a modified set of energy delivery settings and/or other treatment settings are determined, such as automatically by system 10 and/or manually by an operator of system 10 (e.g. the patient and/or a health professional of the patient). In some embodiments, a modified set of treatment settings comprise stopping the delivery of energy by device 100/200 (e.g. turning off stimulation). In some embodiments, a modified new set of energy delivery settings and/or other treatment settings are determined based on the one or more patient parameters recorded in STEP 2020 and/or previous data collected using system 10 (e.g. for the current patient and/or a population of other patients). For example, algorithm 50 (e.g. an AI algorithm) can determine a new set of treatment settings based on parameters selected from the group consisting of: current patient parameters; previous patient parameters (e.g. parameters from the same patient and/or one, two, or more different patients treated using system 10); change in patient parameters (e.g. magnitude of change of patient parameters); current energy delivery settings; previous energy delivery settings (e.g. previous energy delivery parameters used with the same patient and/or one, two, or more different patients treated using system 10); and combinations of these. Once the new energy delivery settings and/or other treatment settings are determined, therapy is provided with those settings (e.g. energy is delivered at those settings), and method 2000 continues with the monitoring of patient parameters of STEP 2020, and method 2000 cyclically repeats.

In some embodiments, delivery of energy by device 100/200 in method 2000 can be stopped at any time (e.g. by the patient via a control on a user interface of system 10, and/or by system 10 such as when a system parameter or patient parameter exceeds a threshold).

In some embodiments, system 10 is configured to modify a treatment setting comprising a non-energy delivery setting when a threshold is exceeded as determined in STEP 2030, such as by performing an action selected from the group consisting of: wake up the patient (e.g. via an audio or tactile alert provided by a functional element 999 and/or consumer device 940 comprising a cell phone, a tablet, a computer, an alarm clock, a bed shaker, and/or other alert-capable device); change the patient's position (e.g. via an adjustable bed and/or other robotic patient manipulator); change the patient's environment (adjust a shade, air conditioning, heat, and/or sound device); and combinations of these. One or more of these actions can be performed in STEP 2030 (e.g. with or without also modifying energy delivery settings).

In some embodiments, algorithm 50 is configured to analyze sounds produced by the patient during sleep (e.g. snoring and/or other breathing sounds) via one or more sensors of system 10. In these embodiments, algorithm 50 can be configured to differentiate the patient's sounds (e.g. snoring) from other sounds in the room (e.g. sounds from humans or other animals in the room, or sounds from a television) that may also be recorded by the sensor, such as to base any comparisons to a threshold performed in STEP 2030 on snoring signals received only from the patient using system 10. In some embodiments, system 10 comprises two or more sensors that are used to record sounds emitted by the patient, and algorithm 50 differentiates the particular patient's sounds based on: the positioning of each sensor; timing of signals received (e.g. if the same snoring or other sound signal is received at different times by the two sensors, the sensor which received the signal first is closer to source of the sound); and/or other parameters of the multiple sensors recording the snoring and other sleep sounds. In some embodiments, the sensors comprise a sensor included in a functional element 999 comprising one, two, or more cell phones and/or other consumer electronics capable of recording sound. In some embodiments, algorithm 50 is configured to perform various signal processing techniques on sound recordings, such as noise cancellation.

In some embodiments, system 10 comprises sets of multiple similar components (e.g. multiple devices 100/200 and/or multiple functional elements 999 configured to record sleep sounds), and system 10 is configured to be used to treat multiple patients that reside (e.g. at least during sleep) in a single room (e.g. a single bedroom, hospital room, and/or barracks). For example, each patient of the multiple patients can have a device 100/200 implanted in them or otherwise positioned to deliver energy (e.g. stimulation energy) during sleep. In these embodiments, algorithm 50 can be configured to differentiate patient parameters (e.g. sleep sounds) based on the patient for whom the parameter applies, such as to differentiate sounds based on use of multiple sensors as described hereabove.

Referring now to FIG. 3, a flow chart of a method for treating a patient is illustrated, consistent with the present inventive concepts. Method 3000 comprises multiple steps of a patient's treatment, such as steps that include the performance of a diagnostic procedure and/or a treatment procedure. Method 3000 of FIG. 3 is described using system 10 of the present inventive concepts and its components.

In STEP 3010, a patient diagnostic procedure is performed, such as a diagnostic procedure performed with one or more devices of system 10 and configured to determine a treatment plan for a patient with sleep apnea and/or another medical condition. The treatment plan can be determined by a clinician (e.g. an operator of system 10), and/or via algorithm 50 of system 10, such as an AI algorithm that utilizes diagnostic data gathered in STEP 3010. The diagnostic procedure can comprise obtaining a patient history. The diagnostic data can include data collected through the performance of a procedure selected from the group consisting of: polysomnography procedure; a weight and/or body mass index determining procedure.

In STEP 3020, a first patient treatment procedure is performed, where the first treatment is part of the treatment plan determined in STEP 3010. The first patient treatment procedure can comprise a tissue reduction procedure and/or a tissue enhancement procedure, each as described herein. The first patient treatment can comprise one or more treatments performed using system 10 or otherwise, such as a nasal procedure (e.g. adenoidectomy, turbinate reduction, septoplasty, and/or DOME procedure); a soft palate procedure (e.g. a tonsillectomy or tonsil volume reduction, a pharyngoplasty, stimulation of tissue via EDD 100 and/or CEDD 200, and/or implantation of an implant such as a treatment device 930 comprising a soft palate implant and/or FAD 300, each as described herein), and/or a tongue procedure (e.g. a lingual tonsillectomy, advancement of the genioglossus or other tongue muscle, a stimulation of tissue via EDD 100 and/or CEDD 200, a force applying procedure via FAD 300).

In STEP 3030, an optional step can be performed, in which a second patient diagnostic procedure is performed. In embodiments in which STEP 3030 is performed, the treatment plan determined in STEP 3010 can be confirmed, or it can be modified (e.g. based on the additional diagnostic data collected in STEP 3030, such as diagnostic data that is based on the treatment performed in STEP 3020).

In STEP 3040, a second patient treatment procedure is performed, based on the treatment plan of STEP 3010 and/or the modified treatment plan of STEP 3030.

In some embodiments, method 3000 of FIG. 3 is performed on a sleep apnea patient, and includes the performance of at least two, at least three, or all four of the following tissue treatment procedures: tonsil tissue volume reduction; tongue tissue volume reduction; airway muscle tightening, strengthening, and/or toning; and stimulation of one or more nerves and/or muscles of the patient's airway. In some embodiments, adenoid tissue of the patient is treated. The tissue treatment procedures can be performed using one, two, or more of each of devices 100, 200, and/or 300.

In some embodiments, method 3000 includes additional treatments, such as a third, fourth, and so on, such as an additional treatment including implantation and subsequent use of CEDD 200 (e.g. for stimulation of airway tissue) and/or FAD 300 (e.g. for application of a force to airway tissue), each as described herein.

In some embodiments, method 3000 includes the introduction of EDM 150, 250, and/or 350 of devices 100/200/300 through a nostril of the patient, such as to treat adenoid tissue and/or other airway tissue of the patent, as described herein.

In some embodiments, method 3000 includes treating adipocytes and/or other tissue of the tongue, such as using a transoral approach and/or access via the submental space.

In some embodiments, method 3000 includes transfacial energy delivery, as described herein, such as to perform a DOME procedure without lifting the skin of the face.

In some embodiments, the first treatment of STEP 3020 is performed in a first type of hospital setting (e.g. a “doctor's office”), and the second treatment of STEP 3040 is performed in a second type of hospital setting, different than the first setting (e.g. an outpatient or other hospital setting).

In some embodiments, the second treatment of STEP 3040 comprises a DOME procedure (e.g. a DOME procedure performed using EDD 100 of system 10). In these embodiments, a third treatment can be performed such as a procedure including implantation and subsequent use of CEDD 200 (e.g. for stimulation of airway tissue) and/or FAD 300 (e.g. for application of a force to airway tissue), each as described herein.

In some embodiments, the first treatment of STEP 3020 comprises at least a volume reduction of tonsil tissue, and the second treatment of STEP 3040 comprises stimulation of airway tissue (e.g. using an implanted CEDD 200).

In some embodiments, the patient is being treated with CPAP prior to the performance of the first treatment procedure of STEP 3020, and the subsequent treatment procedure (e.g. the second treatment procedure of STEP 3040) includes the patient avoiding the need for CPAP, or using CPAP but at a lower pressure and/or velocity level than that which was used by the patient prior to the performance of the earlier treatment procedures (e.g. the first or other previous treatment procedure). In other words, CPAP is avoided or reduced due to an airway improvement and/or other therapeutic benefit provided in a previous treatment procedure. In some embodiments, use of CEDD 200 (e.g. to stimulate airway tissue) and/or FAD 300 (e.g. to apply a scaffolding force to airway tissue) results in avoidance and/or reduced levels of CPAP for the patient.

Referring now to FIG. 4, a side sectional anatomic view of a chronic energy delivery device implanted in a patient for stimulating a nerve is illustrated, consistent with the present inventive concepts. CEDD 200 of FIG. 4 can be of similar construction and arrangement, and include similar components, as CEDD 200 described in reference to FIG. 1 and/or otherwise herein. CEDD 200 of FIG. 4 comprises an implantable portion 2100 that has been implanted in the patient, and an external portion 2500 that has been positioned (e.g. removably positioned) on the patient's skin, proximate the implantation location of implantable portion 2100. Implantable portion 2100 comprises wrap 2110 (e.g. a flexible sheet and/or tubular structure) which has been implanted to surround (e.g. partially surround as shown) nerve N1. Wrap 2110 can comprise a flexible material such as polyvinylidene fluoride and/or polyvinylidene difluoride. Implantable portion 2100 further comprises one, two, or more electrodes, electrodes 2120 (six shown positioned on wrap 2110). Implantable portion 2100 further comprises one, two, or more ultrasound transducers, UST 2199 (three shown positioned on wrap 2110). UST 2199 can comprise one or more piezo transducers, one or more CMUTs, and/or at least one piezo transducer and at least one CMUT. In some embodiments, wrap 2110 comprises a piezo film in which UST 2199 is integral. UST 2199 can comprise an array of multiple piezo and/or other ultrasound transducers that are arranged to allow receiving of ultrasound energy from multiple directions (e.g. an omnidirectional arrangement of transducers).

External portion 2500 comprises a housing, housing 2501, which surrounds an energy delivery module, EDM 2510. EDM 2510 can be of similar construction and arrangement as EDM 250 described herein. EDM 2510 can comprise an array of one, two, or more ultrasound transducers, UST 2599 (26 elements in an array of 2 rows of 13 elements shown) that are configured to deliver ultrasound energy to UST 2199 of implantable portion 2100. USTs 2599 can comprise one or more piezo transducers, one or more CMUTs, and/or at least one piezo transducer and at least one CMUT.

During therapy delivery, EDM 2510 delivers ultrasound energy to UST 2199, which is converted to electrical energy by UST 2199. Electrodes 2120 receive the electrical energy from UST 2199, via conduits 2121 shown, and electrodes 2120 deliver this electrical energy (e.g. via an applied voltage) to target tissue (e.g. nerve N1 shown). In this configuration, the waveform of the electrical energy delivered to target tissue is dependent on the energy delivery waveform provided by EDM 2510. In some embodiments, implantable portion 2100 comprises a controller (e.g. not shown but of similar construction and arrangement to controller 210 of CEDD 200), such that the electrical energy produced by UST 2199 can be used to generate one or more various stimulation waveforms for delivery by electrodes 2120 to target tissue (e.g. stimulation waveforms that are independent of the energy delivery pattern provided by external portion 2500 to implantable portion 2100).

In some embodiments, external portion 2500 comprises spacer 251 shown, such as spacer 251 described herein. In some embodiments, implantable portion 2100 comprises at least two discrete implantable portions, such as implantable portion 2100 shown (positioned proximate nerve N1), and a second implantable portion 2100′, not shown, but positioned proximate a different segment of nerve N1 or positioned proximate a different nerve (e.g. a different nerve serving a similar role or function) such as is described in reference to FIG. 5 herein.

In some embodiments, CEDD 200 of FIG. 4 is configured to operate in a closed-loop mode, such as is described in reference to FIG. 1, FIG. 2, and otherwise herein. For example, controller 210 (e.g. including algorithm 50) can adjust energy delivery in a closed-loop mode. In some embodiments, at least a portion of controller 210 is included in implantable portion 2100, such that energy delivered by implantable portion 2100 can be adjusted in a closed-loop mode without control signals being sent from an external component of system 10. Controller 210 can comprise a battery, capacitor, and/or other energy storage elements such that electrical energy can be stored by implantable portion 2100. In some embodiments, energy is transferred to an implanted energy storage element, such as via transmission of electrical energy (e.g. via inductive coupling, capacitive coupling, radiofrequency energy delivery, and the like), and/or via transmission of ultrasound energy (e.g. ultrasound energy that is received by one or more ultrasound transducers and converted to electrical energy, as described herein). Controller 210 can comprise a voltage-controlled oscillator that controls the voltage of electrical stimulation energy delivered by electrodes 2120.

Referring now to FIG. 5, a side sectional anatomic view of a chronic energy delivery device implanted in a patient for stimulating a nerve is illustrated, consistent with the present inventive concepts. CEDD 200 of FIG. 5 can be of similar construction and arrangement, and include similar components, as CEDD 200 described in reference to FIG. 1 and/or otherwise herein. CEDD 200 of FIG. 5 comprises a first implantable portion 2100a that has been implanted in the patient, a second implantable portion 2100b that has also been implanted in the patient, and an external portion 2500 that has been positioned (e.g. removably positioned) on the patient's skin, proximate the second implantable portion 2100b. First implantable portion 2100a comprises wrap 2110 which has been implanted to surround (e.g. partially surround as shown) nerve N1. Alternatively, wrap 2110 can simply be positioned proximate and alongside nerve N1 (e.g. when wrap 2110 comprises a tubular structure, such as a flexible tubular structure comprising an array of ultrasound transducers). First implantable portion 2100a further comprises one, two, or more electrodes, electrodes 2120 (six shown positioned on wrap 2110). Second implantable portion 2100b comprises one, two, or more ultrasound transducers, UST 2199, shown contained within a housing, housing 2101. UST 2199 can comprise one or more piezo transducers, one or more CMUTs, and/or at least one piezo transducer and at least one CMUT. In some embodiments, wrap 2110 comprises a piezo film in which UST 2199 is integral. UST 2199 is electrically connected to electrodes 2120 via conduit 2121 (e.g. one, two, or more wires). UST 2199 can comprise an array of multiple piezo and/or other ultrasound transducers that are arranged to allow receiving of ultrasound energy from multiple directions (e.g. an omnidirectional arrangement of transducers).

External portion 2500 of FIG. 5 can be of similar construction and arrangement, and include similar components, as external portion 2500 of FIG. 4 described hereabove.

During therapy delivery, EDM 2510 delivers ultrasound energy to UST 2199, which is converted to electrical energy by UST 2199. Electrodes 2120 receive the electrical energy from UST 2199 via conduit 2121, and electrodes 2120 deliver this electrical energy to target tissue (e.g. nerve N1 shown). In this configuration, the waveform of the electrical energy delivered to target tissue is dependent on the energy delivery waveform provided by EDM 2510. In some embodiments, implantable portion 2100 comprises a controller (e.g. not shown but of similar construction and arrangement to controller 210 of CEDD 200), such that the electrical energy produced by UST 2199 can be used to generate one or more various stimulation waveforms for delivery by electrodes 2120 to target tissue (e.g. stimulation waveforms that are independent of the energy delivery pattern provided by external portion 2500 to implantable portion 2100).

In some embodiments, external portion 2500 comprises spacer 251 shown. Spacer 251 can be of similar construction and arrangement as spacer 251 described in reference to FIG. 4 and otherwise herein. In some embodiments, CEDD 200 comprises a third implantable portion, portion 2100a′ shown, which can be of similar construction and arrangement as first implantable portion 2100a. Third implantable portion 2100a′ can include wrap 2110′ which been implanted to surround (e.g. partially surround as shown) nerve N2 (e.g. when wrap 2110′ comprises a tubular structure, such as a flexible tubular structure comprising an array of ultrasound transducers). Third implantable portion 2100a′ can similarly include one, two, or more electrodes, electrodes 2120′ (six shown positioned on wrap 2110). In some embodiments, wraps 2110 and/or 2110′ comprise a piezo film in which a UST 2199 is integral (e.g. to either or both of wraps 2110 and 2110′). Similar to electrodes 2120 of first implantable portion 2100a, during therapy electrodes 2120′ of third implantable portion 2100a′ can receive electrical energy from UST 2199 via conduit 2121, and these electrodes 2120′ can deliver this electrical energy to tissue, such as nerve N2 shown. Nerve N2 can comprise a nerve serving a similar role or function as nerve N1. In some embodiments, nerve N2 comprises a different segment of the same nerve N1.

In some embodiments, CEDD 200 of FIG. 5 is configured to operate in a closed-loop mode, such as is described in reference to FIG. 1, FIG. 2, and otherwise herein. For example, controller 210 (e.g. including algorithm 50) can adjust energy delivery in a closed-loop mode. In some embodiments, at least a portion of controller 210 is included in implantable portion 2100a and/or 2100b, such that energy delivered by implantable portion 2100a can be adjusted in a closed-loop mode without control signals being sent from an external component of system 10. Controller 210 can comprise a battery, capacitor, and/or other energy storage elements such that electrical energy can be stored by implantable portion 2100. Controller 210 can comprise a voltage-controlled oscillator that controls the voltage of electrical stimulation energy delivered by electrodes 2120.

Referring now to FIGS. 6A-B, side sectional anatomic views of a force-applying device implanted in a patient for applying force to tissue is illustrated, consistent with the present inventive concepts. FAD 300 of FIGS. 6A-B can be of similar construction and arrangement, and include similar components, as FAD 300 described in reference to FIG. 1 and/or otherwise herein. FAD 300 of FIGS. 6A-B has been secured to a first tissue location (e.g. bone B1 as shown) such as to apply a force to a second tissue location (e.g. target tissue, such as muscle Ml as shown). FAD 300 comprises a substrate 3110, with a first portion 3110a that is anchored to tissue using securing elements 3111 (e.g. bone anchors, sutures, staples, and the like). Substrate 3110 comprises a second portion 3110b, that is configured to deflect such as to apply a force upon target tissue. In FIG. 6A, second portion 3110b is shown in a non-deflected state, and in FIG. 6B, second portion 3110b is shown in a deflected state (e.g. angularly deflecting as compared with the condition of FIG. 6A). As described herein, FAD 300 can be configured to apply a force to target tissue to modify properties of the tissue (e.g. strengthen muscle tissue), and/or it can apply a force to cause an increase in cross-sectional area of an airway.

Substrate 3110 can comprise an electromechanical assembly configured to change shape, such as when a voltage, temperature change, and/or other drive signal is supplied. Substrate 3110 can comprise a piezo-electric film (e.g. a bimorph and/or unimorph), a shaped memory metal and/or shaped memory polymer, and/or an electromechanical assembly comprising motors, gears, actuators, cams, and/or other components that can be remotely controlled such as to transition between an undeployed state and a deployed (e.g. force-applying) state. In some embodiments, substrate 3110 can comprise a Peltier element.

Referring now to FIG. 7, a partially transparent anatomic view of a force-applying device implanted in a patient for applying force to tissue is illustrated, consistent with the present inventive concepts. FAD 300 of FIG. 7 can be of similar construction and arrangement, and include similar components, as FAD 300 described in reference to FIG. 1 and/or otherwise herein. FAD 300 of FIG. 7 comprises substrate 3110 which has been implanted in a patient's airway as shown, and includes middle portion 3110d, and end portions 3110c and 3110e. Either or both of portions 3110c and 3110e can be configured to apply (e.g. singly or in combination) a force to opposing sides of an airway, such as is shown in FIG. 7. The force can be applied by FAD 300 in a continuous and/or intermittent fashion, with a constant and/or varied level of force applied to the tissue. The force can be applied in order to strengthen adjacent muscles, and/or scaffold the airway.

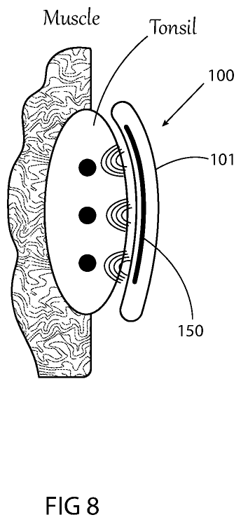

Referring now to FIG. 8, a sectional anatomic view of an energy delivery device delivering energy to tissue is illustrated, consistent with the present inventive concepts. EDD 100 of FIG. 8 can be of similar construction and arrangement, and include similar components, as EDD 100 described in reference to FIG. 1 and/or otherwise herein. EDD 100 of FIG. 8 comprises a housing 101 that surrounds EDM 150 as shown. EDM 150 can comprise an array of ultrasound-based energy delivery elements (e.g. CMUT elements) configured to deliver focused ultrasound (e.g. HIFU) to one or more tissue targets (3 shown) of a patient's tonsil (e.g. a tonsil embedded in other tissue), while avoiding delivery of energy to tissue beneath and/or alongside the tonsil (e.g. muscle tissue beneath and/or alongside the tonsil).

Referring now to FIG. 9, a side view of an energy delivery device delivering energy to tissue captured by the device is illustrated, consistent with the present inventive concepts. EDD 100 of FIG. 9 can be of similar construction and arrangement, and include similar components, as EDD 100 described in reference to FIG. 1 and/or otherwise herein. EDD 100 of FIG. 9 comprises a housing 101 that includes a cup-shaped capture portion 102 that is fluidly attached to a channel, channel 103 shown. Housing 101 can comprise a full (as shown) or partial circumferential geometry. EDD 100 is configured to operably attach, via cable 501, to a console 500 (not shown but such as is described herein), such that console 500 can apply a vacuum via cable 501 (e.g. a vacuum lumen of cable 501) and channel 103 to cause tissue proximate portion 102 to be drawn into portion 102 (tissue T1 as shown). EDD 100 further comprises EDM 150, which can comprise an array of energy delivery elements (e.g. a curved array of energy delivery elements as shown in FIG. 9). EDM 150 can receive energy (e.g. electrical energy) from console 500 via cable 501 (e.g. via one or more wires of cable 501), and subsequently deliver energy (e.g. HIFU or other ultrasound energy) to tissue T1 captured within portion 102. Tissue T1 can comprise tissue positioned within an airway of a patient, such as tonsil tissue and/or other tissue that is comprising an airway of the patient (e.g. and is causing sleep apnea events for the patient).

Referring now to FIG. 10, a perspective view of an energy delivery device delivering energy to tissue captured by the device is illustrated, consistent with the present inventive concepts. EDD 100 of FIG. 10 can be of similar construction and arrangement, and include similar components, as EDD 100 described in reference to FIG. 1 and/or otherwise herein. EDD 100 of FIG. 10 comprises a housing 101 that includes a cone-shaped capture portion 102 that is fluidly attached to a channel, channel 103 shown. Housing 101 can comprise a full (as shown) or partial circumferential geometry. In some embodiments, capture portion 102 comprises a tube-shaped capture portion. EDD 100 is configured to operably attach, via cable 501, to a console 500 (both not shown but such as is described herein), such that console 500 can apply a vacuum via cable 501 (e.g. a vacuum lumen of cable 501) and channel 103 to cause tissue proximate portion 102 to be drawn into the cone-shaped geometry of portion 102. EDD 100 further comprises EDM 150, which can comprise an array of energy delivery elements (e.g. a partial as shown, or full circumferential array of energy delivery elements). EDM 150 can receive energy (e.g. electrical energy) from console 500 via cable 501 (e.g. via one or more wires of cable 501), and subsequently deliver energy (e.g. HIFU or other ultrasound energy) to tissue captured within portion 102. In some embodiments, EDM 150 comprises a full or near full circumferential (e.g. greater than 270°) array of energy delivery elements, such that energy (e.g. HIFU or other ultrasound energy) can be delivered to tissue captured within portion 102 from a full or near full circumferential set of energy delivery directions. Tissue captured within portion 102 can comprise tissue positioned within an airway of a patient, such as tonsil tissue and/or other airway tissue (e.g. tissue that is causing sleep apnea events for the patient). Tissue captured within portion 102 can comprise tissue from one or more various anatomical locations, such as tissue that is part of an organ or other body tissue.

Referring now to FIG. 11, a perspective view of an energy delivery device delivering energy to tissue captured by the device is illustrated, consistent with the present inventive concepts. EDD 100 of FIG. 11 can be of similar construction and arrangement, and include similar components, as EDD 100 described in reference to FIG. 1 and/or otherwise herein. EDD 100 of FIG. 11 comprises a housing 101 that includes a cylinder-shaped capture portion 102. Housing 101 can comprise a full (as shown) or partial circumferential geometry. EDD 100 is configured to operably attach, via cable 501, to a console 500 (not shown but such as is described herein). Capture portion 102 can be slid over tissue to be treated (e.g. tonsil or other tissue). EDD 100 further comprises EDM 150, which can comprise an array of energy delivery elements (e.g. a full, as shown, or partial, circumferential array of energy delivery elements). EDM 150 can receive energy (e.g. electrical energy) from console 500 via cable 501 (e.g. via one or more wires of cable 501), and subsequently deliver energy (e.g. HIFU or other ultrasound energy) to tissue captured within portion 102. In some embodiments, EDM 150 comprises a full or near full circumferential (e.g. greater than 270°) array of energy delivery elements, such that energy (e.g. HIFU or other ultrasound energy) can be delivered to captured tissue from a full or near full circumferential set of energy delivery directions. Tissue to be captured within portion 102 and subsequently treated by EDM 150 can comprise tissue positioned within an airway of a patient, such as tonsil tissue and/or other airway tissue (e.g. tissue that is causing sleep apnea events for the patient). Tissue drawn into capture portion 102 can comprise tissue from one or more various anatomical locations, such as tissue that is part of an organ or other body tissue.

Referring now to FIG. 12, a side sectional anatomic view of an energy delivery device which is positioned on the skin under the patient's chin and delivering energy to tongue tissue is illustrated, consistent with the present inventive concepts. EDD 100 of FIG. 12 can be of similar construction and arrangement, and include similar components, as EDD 100 described in reference to FIG. 1 and/or otherwise herein. EDD 100 of FIG. 12 comprises EDM 150 which can comprise an array of energy delivery elements configured to deliver energy to tissue. In some embodiments, EDD 100 is configured to be positioned at a location L100 that is on the skin, under the chin of the patient, while delivering energy (e.g. HIFU and/or other ultrasound energy) to the patient's tongue and/or other airway tissue of the patient. EDM 150 can be configured to deliver energy to the tongue to reduce the volume of the tongue and/or provide another tissue treatment as described herein. EDM 150 can be configured to be operably attached to a source of energy (e.g. attached to console 500 via cable 501 both not shown but described in detail herein). In some embodiments, EDM 150 can comprise an internal source of power (e.g. functional element 199 comprising a battery and/or other power supply components), and/or a controller (controller 110 described herein) for providing drive signals to EDM 150. In some embodiments, a tool 950 comprising a stabilization device is included such as to attach EDD 100 to the patient during energy delivery. In some embodiments, EDD 100 comprises a spacer positioned between EDM 150 and the patient's skin, such as spacer 151 described herein.

In some embodiments, EDD 100 of FIG. 12 is configured to be positioned on the skin, under the chin of the patient, in order to image tissue of the patient's tongue and/or other airway tissue of the patient (e.g. when also performing an ablation procedure as described hereabove or simply to create image data ID of the patient's tongue or other airway tissue).

Referring now to FIG. 13, a side sectional anatomic view of an energy delivery device that has been advanced transnasally to position a transducer in the patient's airway is illustrated, consistent with the present inventive concepts. EDD 100 of FIG. 13 can be of similar construction and arrangement, and include similar components, as EDD 100 described in reference to FIG. 1 and/or otherwise herein. EDD 100 of FIG. 13 comprises a catheter-like geometry as shown, which can be configured to introduce EDM 150 into the patient's airway through a nostril of the patient (i.e. an intranasal approach). EDM 150 can be configured to deliver energy (e.g. ultrasound energy) to tissue such as to treat (e.g. reduce the volume of and/or stiffen) the tongue, soft palate tissue, tonsil tissue, and/or other airway tissue of the patient.

Referring now to FIG. 14, a front anatomic view of an energy delivery device that has been positioned on the face of a patient is illustrated, consistent with the present inventive concepts. EDD 100 of FIG. 14 can be of similar construction and arrangement, and include similar components, as EDD 100 described in reference to FIG. 1 and/or otherwise herein. EDD 100 of FIG. 14 comprises multiple devices, first device 100a and second device 100b. Device 100a can comprise a tissue contacting geometry (e.g. a rectangle with an aspect ratio of approximately 1) that is different than the tissue contacting geometry of device 100b (e.g. a rectangle with an aspect ratio greater than 1.5 as shown), such as when EDD 100 comprises multiple devices with different tissue contacting geometries configured to accommodate different locations on the patient (e.g. different locations on the patient's face as shown).

In some embodiments, EDD 100 is configured to allow an operator to perform maxillofacial surgery on a patient.

Referring now to FIG. 15, a perspective view of a system including an energy delivery device with a shaft and a distally placed transducer whose diameter approximates that of the shaft is illustrated, consistent with the present inventive concepts. EDD 100 of FIG. 15 can be of similar construction and arrangement, and include similar components, as EDD 100 described in reference to FIG. 1 and/or otherwise herein. EDD 100 of FIG. 15 comprises EDM 150 positioned on the distal end of an elongate housing 101 (e.g. pencil-shaped as shown). The diameter of EDM 150 approximates the diameter of housing 101 (diameter D1 as shown), such that EDM 150 can be introduced through passageways that are as small as diameter D1. The diameter of EDM 150 can be configured for insertion of the EDM 150 through a nostril of the patient (e.g. a diameter of less than 10 mm, or less than 6 mm). EDD 100 can comprise one or more controls, such as a functional element 199 comprising a button as shown (e.g. configured as an on-off control). EDD 100 is operably attached to console 500 via cable 501 as shown. Console 500 comprises user interface 590, functional element 599, and algorithm 50, each as described herein.

Referring now to FIG. 16, a perspective view of a system including an energy delivery device with a shaft and a distally placed transducer whose diameter is larger than that of the shaft is illustrated, consistent with the present inventive concepts. EDD 100 of FIG. 16 can be of similar construction and arrangement, and include similar components, as EDD 100 described in reference to FIG. 1 and/or otherwise herein. EDD 100 of FIG. 16 comprises EDM 150 positioned on the distal end of an elongate housing 101 (e.g. pencil-shaped as shown). The diameter of EDM 150 (diameter D2 as shown) is larger than the diameter of housing 101 (diameter D1 as shown), such that EDM 150 can include a large array of energy delivery elements (e.g. a large array of piezo and/or CMUT ultrasound energy delivery elements). EDD 100 can comprise one or more controls, such as a functional element 199 comprising a button as shown (e.g. configured as an on-off control). EDD 100 is operably attached to console 500 via cable 501 as shown. Console 500 comprises user interface 590, functional element 599, and algorithm 50, each as described herein.

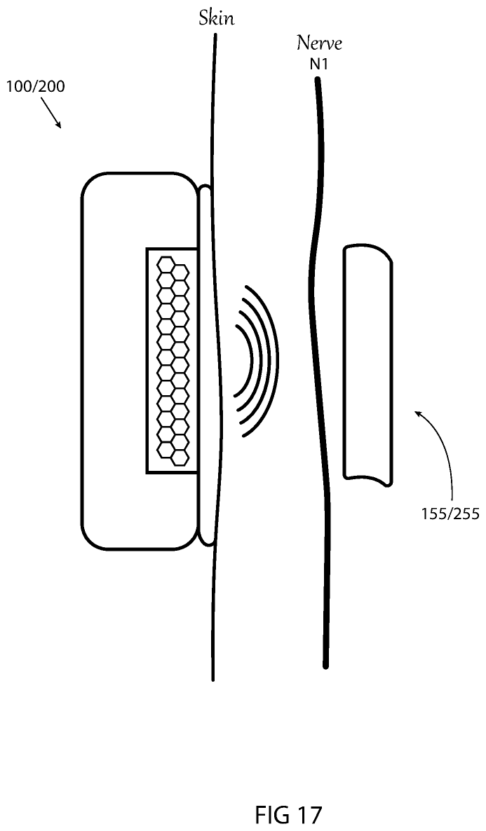

Referring now to FIG. 17, a side sectional anatomic view of an energy delivery device comprising an energy delivery module and a mirror is illustrated, consistent with the present inventive concepts. The energy delivery device of FIG. 17 can comprise a EDD 100 (e.g. a clinician device used in a clinical procedure), and/or CEDD 200 (e.g. an implant or externally placed device used with a patient for an extended period of time, as described herein). EDD 100 and/or CEDD 200 of FIG. 17 can be of similar construction and arrangement, and include similar components, as EDD 100 and/or CEDD 200, respectively, described in reference to FIG. 1 and/or otherwise herein. EDD 100 and/or CEDD 200 (singly or collectively “device 100/200” herein) of FIG. 17 can comprise an energy delivery module, EDM 150/250 (not shown but integral to EDM 150/250) comprising one or more energy delivery elements, such as one or more piezo transducers and/or CMUTs configured to deliver ultrasound energy (e.g. HIFU or other ultrasound energy) to target tissue. Device 100/200 can include mirror 155/255 as shown and described herein. Mirror 155/255 (e.g. an acoustic mirror) can be positioned (e.g. implanted) proximate target tissue (e.g. nerve N1 shown). Mirror 155/255 and EDM 150/250 are positioned (as shown) on opposite sides of the target tissue, such that energy delivered by EDM 150/250 that reaches mirror 155/255 is reflected back toward the target tissue. In some embodiments, EDM 150/250 is positioned on the skin, and mirror 155/255 is implanted beneath the target tissue (e.g. beneath nerve N1 such that N1 is positioned between EDM 150/250 and mirror 155/255). In other embodiments, both EDM 150/250 and mirror 155/255 are implanted in the patient (e.g. on opposite sides of target tissue to be treated).

Referring now to FIGS. 18A-B, a top view, and a side sectional anatomic view of an energy delivery device are illustrated, consistent with the present inventive concepts. EDD 100 of FIGS. 18A-B can be of similar construction and arrangement, and include similar components, as EDD 100 described in reference to FIG. 1 and/or otherwise herein. EDD 100 of FIGS. 18A-B comprises an energy delivery module, EDM 150, with a spoon-like geometry. EDM 150 can comprise a curved geometry along one axis (as shown in FIG. 18B) or along multiple axes. EDM 150 can comprise one or more piezo transducers and/or one or more CMUTs configured to deliver ultrasound energy. In FIGS. 18A-B, EDM 150 has been inserted through a patient's mouth, and positioned on the patient's tongue, such as to subsequently deliver energy to ablate and/or otherwise treat tissue of the tongue, as described herein.

Referring now to FIG. 19, a side sectional anatomic view of an energy delivery device that has been transnasally inserted into a patient is illustrated, consistent with the present inventive concepts. EDD 100 of FIG. 19 can be of similar construction and arrangement, and include similar components, as EDD 100 described in reference to FIG. 1 and/or otherwise herein. EDD 100 of FIG. 19 comprises a first portion 100a comprising an energy delivery module EDM 150, on its distal portion, and a second portion 100b comprising an acoustic mirror, mirror 155, on its distal portion, all as shown. EDM 150a comprises one or more piezo transducers and/or one or more CMUTs configured to deliver ultrasound energy (e.g. HIFU and/or other ultrasound energy). As shown in FIG. 19, the distal portion of EDD 100a has been inserted into the patient's nasal canal via a first nostril, and the distal portion of EDD 100b has been inserted into the patient's nasal canal via the other nostril, such that EDM 150 and mirror 155 have been positioned on opposite sides of a section of the patient's nasal septum S1, at location L100 as shown. EDM 150 can deliver energy to the tissue of location L100 such as to ablate, soften, and/or otherwise treat tissue of the nasal septum (e.g. as described herein). Mirror 155 is configured and positioned to reflect ultrasound energy that passes through the tissue of L100 back onto that tissue. In some embodiments, EDD 100b includes another energy delivery module (e.g. an EDM 150b) positioned at the location of mirror 155 (e.g. instead of mirror 155). In some embodiments, EDD 100 comprises only EDD 100a and is void of EDD 100b. Delivery of ultrasound energy to L100 can allow subsequent reshaping of the patient's nasal canal, such as a reshaping that is facilitated by the softening or other treatment caused by delivery of ultrasound energy to that tissue by EDD 100. In some embodiments, reshaping of the patient's nasal canal is performed via surgery and/or a catheter device including a balloon configured to apply a reshaping force to the patient's nasal canal.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for performing a medical procedure on a patient comprising:
at least one of:
an energy delivery device;
a chronic energy delivery device; and/or
a force applying device,
wherein the system is configured to perform a medical procedure on the patient.

2.-12. (canceled)
